(19) United States
(12) Patent Application Publication
(10) Pub. No.: US 2005/0170397 A1
(43) Pub. Date: Aug. 4, 2005

(54) Novel receptors

(75) Inventors: Hui Tian, Foster City, CA (US); Jiagang Zhao, San Diego, CA (US); Jin-Long Chen, San Mateo, CA (US); Gene Cutler, San Francisco, CA (US); Songzhu An, San Francisco, CA (US); Kang Dai, Albany, CA (US); Jamila S. Gupte, San Mateo, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Tularik Inc., South San Francisco, CA (US)

(21) Appl. No.: 11/038,360

(22) Filed: Jan. 18, 2005

Related U.S. Application Data
(63) Continuation of application No. 09/990,940, filed on Nov. 21, 2001.
(60) Provisional application No. 60/252,841, filed on Nov. 22, 2000.
(60) Provisional application No. 60/261,377, filed on Jan. 12, 2001.
(60) Provisional application No. 60/280,696, filed on Mar. 29, 2001.
(60) Provisional application No. 60/257,636, filed on Dec. 22, 2000.
(60) Provisional application No. 60/279,554, filed on Mar. 28, 2001.

(51) Int. Cl. C12Q 1/68; C07H 21/04; C07K 14/705; C07K 16/28

(52) U.S. Cl. 435/6; 435/69.1; 435/320.1; 435/325; 530/350; 536/23.5; 530/388.22

(57) ABSTRACT

The invention provides isolated nucleic acid and amino acid sequences of novel G-protein coupled receptors, antibodies to such receptors, methods of detecting such nucleic acids and receptors, and methods of screening for modulators of G-protein coupled receptors.

### CROSS-REFERENCES TO RELATED APPLICATIONS

This application claims priority to U.S. Provisional Application No. 60/252,841, filed Nov. 22, 2000; U.S. Provisional Application No. 60/261,377, filed Jan. 12, 2001; U.S. Provisional Application No. 60/280,696, filed Mar. 29, 2001; U.S. Provisional Application No. 60/257,636, filed Dec. 22, 2000; and U.S. Provisional Application No. 60/279,554, filed Mar. 28, 2001, each of which applications is herein incorporated by reference.

### FIELD OF THE INVENTION

The invention provides isolated nucleic acid and amino acid sequences of a novel human G-protein coupled receptor, antibodies to such receptor, methods of detecting such nucleic acid and receptor, and methods of screening for modulators of G-protein coupled receptors.

### BACKGROUND OF THE INVENTION

G-protein coupled receptors are cell surface receptors that indirectly transduce extracellular signals to downstream effectors, which can be intracellular signaling proteins, enzymes, or channels, and changes in the activity of these effectors then mediate subsequent cellular events. The interaction between the receptor and the downstream effector is mediated by a G-protein, a heterotrimeric protein that binds GTP. G-protein coupled receptors (“GPCRs”) typically have seven transmembrane regions, along with an extracellular domain and a cytoplasmic tail at the C-terminus. These receptors form a large superfamily of related receptors molecules that play a key role in many signaling processes, such as sensory and hormonal signal transduction. For example, a large family of olfactory GPCRs has been identified (see, e.g., Buck & Axel, Cell 65:175-187 (1991)). The further identification of GPCRs is important for understanding the normal process of signal transduction and as well as its involvement in pathologic processes. For example, GPCRs can be used for disease diagnosis as well as for drug discovery.

Eating disorders, which represent a major health concern throughout the world, have been linked to GPCR regulation. On the one hand, disorders such as obesity, the excess deposition of fat in the subcutaneous tissues, manifest themselves by an increase in body weight. Individuals who are obese often have, or are susceptible to, medical abnormalities including respiratory difficulties, cardiovascular disease, diabetes and hypertension. On the other hand, disorders like cachexia, the general lack of nutrition and wasting associated with chronic disease and/or emotional disturbance, are associated with a decrease in body weight.

The neuropeptide melanin-concentrating hormone (MCH), a cyclic hypothalmic peptide involved in the regulation of several functions in the brain, has previously been found to be a major regulator of eating behavior and energy homeostasis. It has previously been determined that MCH is the natural ligand for the 353-amino acid orphan G-Protein-Coupled-Receptor (GPCR) termed SLC-1. Subsequent to this determination, SLC-1, which is sequentially homologous to the somatostatin receptors, is frequently referred to as melanin-concentrating hormone receptor (MCH receptor or MCHR) (see, Chambers et al., Nature 400:261-65 (1999); Saito et al., Nature 400:265-69 (1999); and Saito et al., TEM 11(8):299-303 (2000)).

Compelling evidence exists that MCH is involved in regulation of eating behavior. First, intracerebral administration of MCH in rats resulted in stimulation of feeding. Next, mRNA corresponding to the MCH precursor is up-regulated in the hypothalamus of genetically obese mice and of fasted animals. Finally, mice deficient in MCH are leaner and have a decreased food intake relative to normal mice. MCH is believed to exert its activity by binding to MCHR, resulting in the mobilization of intracellular calcium and a concomitant reduction in forskolin-elevated cAMP levels (see, Chambers et al., Nature 400:261-65 (1999); and Shimada et al. Nature 396:670-74 (1998)). MCH also activates inwardly rectifying potassium channels, and MCHR has been found to interact with both Gαi protein and Gαq protein (see, Saito et al., TEM 11(8):299-303 (2000)). Moreover, analysis of the tissue localization of MCHR indicates that it is expressed in those regions of the brain involved in olfactory learning and reinforcement. The cumulative data suggest that modulators of MCHR should have an effect on neuronal regulation of food intake (see, Saito et al., Nature 400:265-69 (1999)).

GPCRs are also involved in retinal function and additionally may play an important role in the pathology of retinal disease. Retinitis pigmentosa is a retinal degeneration characterized by the following manifestations: night blindness, progressive loss of peripheral vision, eventually leading to total blindness; ophthalmoscopic changes consist in dark mosaic-like retinal pigmentation, attenuation of the retinal vessels, waxy pallor of the optic disc, and in the advanced forms, macular degeneration. In some cases there can be a lack of pigmentation. Retinitis pigmentosa can be associated and degenerative opacity of the vitreous body, and cataract. A number of more complex syndromes are often associated to this disease, such as Usher's syndrome, responsible for deafness; Laurence-Moon syndrome, characterized by hypogonadism, mental retardation and obesity; Refsum's syndrome which can lead to mental retardation and dwarfism. Family history is prominent in retinitis pigmentosa; the pattern of inheritance may be autosomal recessive, autosomal dominant, or X-linked; the autosomal recessive form is the most common and can occur sporadically. Disease incidence varies from 1/2000 to 1/7000 according to the type of investigation and geographic location. Although retinitis pigmentosa was first described a century ago; its pathogenesis is, nevertheless, still unknown. (see, Molecular Genetic Investigations of Eye Disease, http://ucl.ac.uk/ioo/research/bhattacharya.htm, and den Hollander, Nature Genetics, 23:217-221 (October 1999), the teachings of both of which are incorporated herein by reference).

The phenomenon of circadian rhythms in biology is well known, and circadian rhythms are exhibited by all eukaryotic plants and animals, including man. Biological rhythms are periodic fluctuations in biological properties over time; these include circadian as well as seasonal variations. Circadian, or approximately 24-hour, rhythms include the production of biological molecules such as hormones, the regulation of body temperature, and behaviors such as wakefulness, sleep and periods of activity. In nature, circadian rhythms are closely tied to environmental cues that impose a 24-hour pattern on many of these fluctuations. Experimental inquiry has established that when these cues are absent, most circadian rhythms have a periodicity of approximately 25 hours. Circadian rhythms that are not regulated by environmental cues are said to be free-running. The regulation of circadian rhythms by signals from the environment is said to involve entrainment of the circadian rhythms. The environmental signals that effect entrainment have been termed zeitgebers, an example of which is the light-dark cycle.

It is thought in the art that the control of circadian rhythms in mammals is mediated by a portion of the brain called the superchiasmatic nuclei (SCN). The hypthalamic SCN receives an array of distinct neurochemical inputs. Among these, the retinohypothalamic tract (RHT) carries light information directly from the retina to the SCN and represents the major nocturnal regulatory pathway. Although a coherent picture of the clock mechanism has recently emerged through molecular genetics, relatively little is known about the chemical messengers, cellular pathways or downstream signaling events that link the daily oscillations of pacemaker cells to overt behavioral rhythms. (See, Renn, et al., Cell, 99:791-802 (Dec. 23, 1999); Chen, et al., Proc. Natl. Acad. Sci., 96(23):13468-13473 (Nov. 9, 1999), the teachings of both of which are incorporated by reference for all purposes.)

Thus there exists a need in the art to identify the genes involved in eating disorders and hypothalamic control; in retinitis pigmentosi, and other pathologies of retinal function; and in the regulation of circadian rhythm; as well as other. Moreover, what is needed in the art are methods for treating these orders. The present invention fulfills these and other needs.

### SUMMARY OF THE INVENTION

The present invention thus provides nucleic acids encoding novel G-protein coupled receptors, methods of detecting such receptors and the nucleic acids encoding them, methods of identifying modulators of such receptors, and methods of diagnosing and treating disease states associated with the receptors or mutants thereof.

In one aspect, the present invention provides an isolated nucleic acid encoding a G-protein coupled receptor polypeptide, the nucleic acid encoding a polypeptide comprising at least about 70% amino acid identity, often 80%, 85%, 90%, or 95% sequence identity, to an amino acid sequence of SEQ ID NO:2, SEQ ID NO:4, SEQ ID NO:6, SEQ ID NO:8, SEQ ID NO:10, SEQ ID NO:12, SEQ ID NO:16, or SEQ ID NO:18. In other embodiments, the nucleic acid comprises the nucleotide sequence of SEQ ID NO:1, SEQ ID NO:3, SEQ ID NO:5, SEQ ID NO:7, SEQ ID NO:9, SEQ ID NO:11, SEQ ID NO:15, or SEQ ID NO:17.

The nucleic acid can encode a polypeptide, which can have G-protein coupled receptor activity, that specifically binds to polyclonal antibodies generated against an amino acid sequence of SEQ ID NO:2, SEQ ID NO:4, SEQ ID NO:6, SEQ ID NO:8, SEQ ID NO:10, SEQ ID NO:12, SEQ ID NO:16, or SEQ ID NO:18. In some embodiments, the polypeptide comprises an amino acid sequence of SEQ ID NO:2, SEQ ID NO:4, SEQ ID NO:6, SEQ ID NO:8, SEQ ID NO:10, SEQ ID NO:12, SEQ ID NO:16, or SEQ ID NO:18.

In other embodiments, the nucleic acid is amplified by primers that specifically hybridize under stringent hybridization conditions to SEQ ID NO:1, SEQ ID NO:3, SEQ ID NO:5, SEQ ID NO:7, SEQ ID NO:9, SEQ ID NO:11, SEQ ID NO:15, or SEQ ID NO:17 and/or can specifically hybridizes under stringent hybridization conditions to a nucleic acid comprising a nucleotide sequence of SEQ ID NO:1, SEQ ID NO:3, SEQ ID NO:5, SEQ ID NO:7, SEQ ID NO:9, SEQ ID NO:11, SEQ ID NO:15, or SEQ ID NO:17.

In another aspect, the invention provides an isolated nucleic acid encoding a polypeptide comprising at least 15, often 20 contiguous amino acids of an amino acid sequence of SEQ ID NO:2, SEQ ID NO:4, SEQ ID NO:6, SEQ ID NO:8, SEQ ID NO:10, SEQ ID NO:12, SEQ ID NO:16, or SEQ ID NO:18.

In another aspect, the invention provides an isolated polypeptide comprising at least 70% amino acid sequence identity to an amino acid sequence of SEQ ID NO:2, SEQ ID NO:4, SEQ ID NO:6, SEQ ID NO:8, SEQ ID NO:10, SEQ ID NO:12, SEQ ID NO:16, or SEQ ID NO:18. The polypeptide that has G-protein coupled receptor activity In some embodiments, the polypeptide comprises 80%, or 90% identity to the amino acid sequence. In other embodiment, the polypeptide comprises an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:16, or SEQ ID NO:18. In other embodiments, the polypeptide specifically binds to polyclonal antibodies generated against an amino acid sequence of SEQ ID NO:2, SEQ ID NO:4, SEQ ID NO:6, SEQ ID NO:8, SEQ ID NO:10, SEQ ID NO:12, SEQ ID NO:16, or SEQ ID NO:18.

In another aspect, the present invention provides an antibody that binds to a polypeptide of the invention.

In another aspect, the present invention provides expression vectors comprising the nucleic acids of the invention, and host cells comprising the expression vectors.

In another aspect, the invention provides a method for identifying a compound that modulates signal transduction, the method comprising: (i) contacting the compound with a polypeptide comprising at least 70% amino acid sequence identity, of ten 80%, 85%, 90%, or 95% amino acid sequence identity, to an amino acid sequence of SEQ ID NO:2, SEQ ID NO:4, SEQ ID NO:6, SEQ ID NO:8, SEQ ID NO:10, SEQ ID NO:12, SEQ ID NO:16, or SEQ ID NO:18; and (ii) determining the functional effect of the compound upon the polypeptide. In one embodiment, the polypeptide comprises the amino acid sequence of SEQ ID NO:2, SEQ ID NO:4, SEQ ID NO:6, SEQ ID NO:8, SEQ ID NO:10, SEQ ID NO:12, SEQ ID NO:16, or SEQ ID NO:18.

Alternatively, the invention provides a method of identifying a compound that modulates signal transduction, the method comprising: (i) contacting the compound with a polypeptide comprising at least 15, often 20 contiguous amino acids of S SEQ ID NO:2, SEQ ID NO:4, SEQ ID NO:6, SEQ ID NO:8, SEQ ID NO:10, SEQ ID NO:12, SEQ ID NO:16, or SEQ ID NO:18; and (ii) determining the functional effect of the compound upon the polypeptide.

In practising the methods, the polypeptide can be linked, e.g., covalently linked, to a solid phase. The functional effect can be a chemical effect or a physical effect. In one embodiment, the functional effect is determined by measuring changes in intracellular cAMP, IP₃, or Ca²⁺. The functional effect can also be determined by measuring binding of the compound to the polypeptide.

In one embodiment, the polypeptide is recombinant and is often expressed in a cell or cell membrane, e.g., a eukaryotic cell or cell membrane. In other embodiments, the eukaryotic cell is a brain cell, e.g., a hypothalamic, or hippocampal cell, a retinal cell, or an adipocyte. The cell can also be from pancreas; kidney; liver; lung; an immune system cell, e.g., from spleen, lymph node, thymus, or bone marrow; or from placenta.

In another aspect, the invention provides a method of identifying a mammal having a TGR-associated disorder, the method comprising detecting a nucleic acid molecule that comprises greater than 70% nucleic acid sequence identity, often 80%, 85%, 90%, or 95% identity, to a sequence of SEQ ID NO:1, SEQ ID NO:3, SEQ ID NO:5, SEQ ID NO:7, or SEQ ID NO:9, in a sample from the mammal, wherein abnormal expression of the nucleic acid molecule in the sample indicates that the mammal has a TGR-associated disorder. In one embodiment, the nucleic acid molecule comprises the sequence of SEQ ID NO:1, SEQ ID NO:3, SEQ ID NO:5, SEQ ID NO:7, or SEQ ID NO:9.

Additionally, the invention provides a method of identifying a mammal having a TGR-associated disorder, the method comprising detecting a nucleic acid molecule that comprises at least 15, often 20, contiguous nucleotides of a nucleic acid sequence of SEQ ID NO:1, SEQ ID NO:3, SEQ ID NO:5, SEQ ID NO:7, or SEQ ID NO:9 in a sample from the mammal, wherein abnormal expression of the nucleic acid molecule in the sample indicates that the mammal has a TGR-associated disorder.

The invention also provides a method of identifying a mammal having a TGR-associated disorder, the method comprising detecting a polypeptide that comprises greater than 70% amino acid sequence identity, often 80%, 85%, 90%, or 95% sequence identity, to an amino acid sequence of SEQ ID NO:2, SEQ ID NO:4, SEQ ID NO:6, SEQ ID NO:8, or SEQ ID NO:10 in a sample from the mammal, wherein abnormal expression of the polypeptide in the sample indicates that the mammal has a TGR-associated disorder. In one embodiment, the polypeptide comprises the amino acid sequence of SEQ ID NO:2, SEQ ID NO:4, SEQ ID NO:6, SEQ ID NO:8, or SEQ ID NO:10.

In addition, the invention provides a method of identifying a mammal having a TGR-associated disorder, the method comprising detecting a polypeptide that comprises at least 15, often 20, contiguous amino acids of an amino acid sequence of SEQ ID NO:2, SEQ ID NO:4, SEQ ID NO:6, SEQ ID NO:8, or SEQ ID NO:10 in a sample from the mammal, wherein abnormal expression of the polypeptide in the sample indicates that the mammal has a TGR-associated disorder.

In another aspect, the invention provides a method of detecting the presence of a TGR-GPCR nucleic acid or polypeptide in human tissue, the method comprising: (i) isolating a biological sample; (ii) contacting the biological sample with a TGR-GPCR-specific reagent that selectively associates with an TGR-GPCR nucleic acid or polypeptide; and, (iii) detecting the level of TGR-GPCR-specific reagent that selectively associates with the sample. In one embodiment, the TGR-GPCR-specific reagent includes, but is not limited to, antibodies, oligonucleotide primers, and nucleic acid probes.

In another aspect, the present invention provides a method of treating a patient with a disease or condition associated with a GPCR activity, comprising administering to the patient a modulator of a GPCR sequence selected from the group consisting of SEQ ID NO:2, SEQ ID NO:4, SEQ ID NO:6, SEQ ID NO:8, or SEQ ID NO:10, SEQ ID NO:16, or SEQ ID NO:18.

In particular embodiments, the present invention provides a method of treating obesity, the method comprising the step of administering to a patient a therapeutically effective amount of a compound that modulates TGR342 or, alternatively, MCHR2. In a preferred embodiment, the compound is identified using the methods of the invention.

In another aspect, the present invention provides a method of treating a disorder including, but not limited to, hyperlipidemia, dyslexia, kidney disease, cardiac myxoma, and cerebral cavernous malformations, the method comprising the step of administering to a patient a therapeutically effective amount of a compound that modulates TGR342 or, alternatively, MCHR2. In a preferred embodiment, the compound is identified using the methods of the invention.

In another aspect, the present invention provides a method of treating a disorder including, but not limited to, sleep disorders, growth disorders, disorders associated with dopaminergic system function and reproductive disorders, the method comprising the step of administering to a patient a therapeutically effective amount of a compound that modulates TGR342 or, alternatively, MCHR2. In a preferred embodiment, the compound that modulates TGR342 or, alternatively, MCHR2. In a preferred embodiment, the compound is identified using the methods of the invention.

In another aspect, the present invention provides a method of identifying a mammal having a TGR342-associated disorder, the method comprising detecting a TGR342 nucleic acid molecule in a sample from the mammal, wherein abnormal expression of the TGR342 nucleic acid molecule in the sample indicates that the mammal has a TGR342-associated disorder. As used herein, abnormal expression refers to, e.g., altered, modified or inappropriate expression, such as over expression, under expression or expression in a cell that does not normally express TGR342, compared to a mammal that does not have a TGR342-associated disorder.

In another aspect, the present invention provides a method of identifying a mammal having a TGR342-associated disorder, the method comprising detecting a TGR342 polypeptide in a sample from the mammal, wherein abnormal expression of the TGR342 polypeptide in the sample indicates that the mammal has a TGR342-associated disorder. As used herein, abnormal expression refers to, e.g., altered, modified or inappropriate expression, such as over expression, under expression or expression in a cell that does not normally express TGR342, compared to a mammal that does not have a TGR342-associated disorder.

In another aspect, the present invention provides a method of treating or preventing a TGR342-associated disorder, the method comprising administering a therapeutically effective amount of a compound that modulates TGR342 to a mammal in need thereof. In a preferred embodiment, the modulator is identified using the methods of the present invention.

### BRIEF DESCRIPTION OF THE DRAWINGS

FIG. 1 sets forth an amino acid sequence alignment between TGR342 (MCHr2), melanin-concentrating hormone (MCHr1) and somatostatin receptor 1 (SSTR1). The arrow indicates the end of the putative truncated form of TGR342 that results from alternative splicing.

FIG. 2 sets forth exemplary expression data for TGR342. FIG. 2A shows a Northern analysis of a multi-tissue blot with β-actin as an internal control. FIG. 2B shows a Northern blot of tissues from various regions of the brain and shows exemplary expression of TGR342 and MCHR1 mRNA in the tissues with glyceraldehyde phosphate dehydrogenase as an internal control. FIG. 2C shows a PCR analysis.

FIG. 3 sets forth a TGR342 signal transduction pathway analysis. FIG. 3A provides the results of a NFAT-luc assay, while FIG. 3B provides the results of a CREB-luc assay.

FIG. 4 sets forth graphs showing the dose response of the activation of TGR342 by MCH using an Aequorin assay.

FIG. 5 sets forth a graph showing specific activation of TGR342 by MCH. MCH or MCH-related peptides were tested for TGR342 agonist activity.

FIG. 6 sets forth exemplary binding data for MCH binding to TGR342. A graph shows the binding of [¹²⁵I]-MCH to membranes prepared from HEK293 cells transiently transfected with a TGR342 expression construct.

FIG. 7 shows exemplary data showing that pertussis toxin (PTX) specifically inhibits Gi signaling.

FIG. 8 shows exemplary data showing that MCH increased the production of inositol phosphate in TGR342-transfected cells in the presence or absence of PTX.

FIG. 9 sets forth exemplary expression data or TGR60.

FIGS. 10A and 10B set forth exemplary expression data for mouse TGR 346a (FIG. 10A) and mouse TGR346b (FIG. 10B).

### DETAILED DESCRIPTION OF THE INVENTION AND PREFERRED EMBODIMENTS

Introduction

The present invention provides for the first time nucleic acids encoding novel G protein coupled receptors. The nucleic acids and the receptors that they encode are referred to individually as TGR342, TGR60, TGR346, and TGR349. These GPCRs are components of signal transduction pathways in a variety of cells. he nucleic acids and the encoded receptors provide, inter alia, valuable probes for the identification of particular cell types, as evidenced by specific patterns of expression, for the isolation of specific modulators of GPCR activity in different cell types, for use as genetic markers, as the chromosomal location of many of them is known, and for the identification of mutations associated with diseases resulting from GPCR inactivation in particular cell types.

Nucleic acids encoding the GPCR of the invention can be identified using techniques such as reverse transcription and amplification of mRNA, isolation of total RNA or poly A⁺ RNA, northern blotting, dot blotting, in situ hybridization, RNase protection, S1 digestion, probing DNA microchip arrays, and the like.

In particular, the invention provides a novel GPCR, TGR342, which has been determined to be a receptor for MCH. This is the second known receptor for MCH, the first being described in International Application No. PCT/US00/15503, hereby incorporated by reference. Thus, modulation of TGR342 can be used to treat or prevent known MCHR-associated conditions. Such conditions are well characterized and include, but are not limited to, weight regulation, diabetes, learning and other neuronal-related functions.

The present invention also provides polymorphic variants of the GPCR depicted in SEQ ID NO:2 (human TGR342): variant #1, in which an isoleucine residue is substituted for a leucine acid residue at amino acid position 15; variant #2, in which an glutamic acid residue is substituted for a aspartic acid residue at amino acid position 33; and variant #3, in which a glycine residue is substituted for an alanine residue at amino acid position 42.

The invention also provides nucleic acids encoding two novel TGR60 GPCRs. The two proteins are generated by alternative splicing of an mRNA. The two nucleic acids and the receptors that they encode are collectively referred to as TGR60. These GPCRs are components of signal transduction pathways in a variety of cells. It has been determined that TGR60 is specifically expressed in the hypothalamus and retina. Vasopressin and oxytocin receptors are the closest vertebrate homologs with about 33% identity to TGR60 at the amino acid level. To date, only a singly oxytocin receptor has been cloned so far, yet pharmacological evidence suggests the existence of a second receptor. There is also evidence indicating the existence of a separate receptor for vasopressin metabolites. TGR60 shares an overall 43% identity to a Drosophila putative GPCR, CG6111 (Celera fly genome project), yet no oxytocin- or vasopressin-like peptides have been reported in fruit fly. It is thought that TGR60 is the second of the oxytocin receptors, and that it is also the receptor for vasopressin metabolites. Moreover, a GeneBank entry AC005853 containing TGR60 sequence has been mapped to chromosome 7p14-15 between markers D7S795-D7S526, where retinitis pigmentosa 9 locus was localized. The mapping data plus the retina-specific expression data indicate that the TGR60 gene is associated with this eye disease. Further, the selective expression of TGR60 in the components of the retinohypothalamic tract (RHT) also indicates a role for this gene in the control of circadian rhythms. Interestingly, in rodents, the expression of vasopressin neuropeptide in the suprachiasmatic nuclei of the hypothalamus is under the influence of the clock. In Drosophila, the pigment-dispensing factor (PDF), i.e., PDF neuropeptide, has been demonstrated to be the principal circadian transmitter. Taken together, this information indicates that TGR60 is the receptor for human PDF or a human PDF-like peptide. As such, the present invention provides methods for treating retinitis pigmentosa by modulating the activity of TGR60 as well as methods for regulating circadian rthyms by modulating the activity of TGR60.

Chromosome localization of several of the genes has been determined, and are localized as follows: human TGR342 maps to chromosome 6; human TGR60 maps to chromosome 7p14-15 between markers D7S795-D7S526; human TGR339 maps to chromosome 7; and human TGR346 maps to chromosome 4. These GPCR gene can be used to identify diseases, mutations, and traits caused by and associated with the GPCRs.

Various aspects of the cell-type specific expression of the present GPCRs have been determined. Human TGR342 has been shown to be expressed in several brain sub-regions including cerebral cortex, frontal lobe, parietal lobe, occipital lobe, temporal lobe, paracentral gyrus of cerebral cortex, amygdala, hippocampus, caudate nucleus, the brain and adipocytes. Such tissue specific expression indicates that the present GPCRs can be used to specifically modulate GPCR activity in particular cell types. In addition, certain diseases or conditions, or a propensity for the diseases or conditions, may be detected by detecting mutations in particular GPCRs, as described infra.

Human TGR60 is specifically expressed in the hypothalamus and retina. Modulaters of TGR60 can be used to treat or prevent TGR60-associated conditions of the hypothalamus and retina.

Human TGR339 is expressed in the brain, kidney, liver, lung, placenta, adipose, spleen, lymph node, thymus, bone marrow and fetal liver; and human TGR346 is expressed in the hypothalamus, hippocampus and kidney.

Such tissue specific expression indicates that the present GPCRs can be used to specifically modulate GPCR activity in particular cell types. In addition, certain diseases or conditions, or a propensity for the diseases or conditions, may be detected by detecting mutations in particular GPCRs, as described infra.

The isolation of novel GPCRs provides a means for assaying for and identifying modulators of G-protein coupled receptor signal transduction, e.g., activators, inhibitors, stimulators, enhancers, agonists, and antagonists. Such modulators of signal transduction are useful for pharmacological modulation of signaling pathways, e.g., in cells such as kidney cells, liver cells, colon cells, hypothalamus cells, neurons, retinal cells, spleen cells, and adipocytes. Such activators and inhibitors identified using GPCRs can also be used to further study signal transduction. Thus, the invention provides assays for signal transduction modulation, where the GPCRs act as direct or indirect reporter molecules for the effect of modulators on signal transduction. GPCRs can be used in assays in vitro, ex vivo, and in vivo, e.g., to measure changes in transcriptional activation of GPCRs; ligand binding; phosphorylation and dephosphorylation; GPCR binding to G-proteins; G-protein activation; regulatory molecule binding; voltage, membrane potential, and conductance changes; ion flux; changes in intracellular second messengers such as cAMP and inositol triphosphate; changes in intracellular calcium levels; and neurotransmitter release.

Methods of assaying for modulators of signal transduction include in vitro ligand binding assays using the GPCRs, portions thereof such as the extracellular domain, or chimeric proteins comprising one or more domains of a GPCR, oocyte GPCR expression or tissue culture cell GPCR expression, either naturally occurring or recombinant; membrane expression of a GPCR, either naturally occurring or recombinant; tissue expression of a GPCR; expression of a GPCR in a transgenic animal, etc.

Functionally, the GPCRs represent a seven transmembrane G-protein coupled receptor of the G-protein coupled receptor family, which interact with a G protein to mediate signal transduction (see, e.g., Fong, Cell Signal 8:217 (1996); Baldwin, Curr. Opin. Cell Biol. 6:180 (1994)).

Specific regions of the GPCR nucleotide and amino acid sequences may be used to identify polymorphic variants, interspecies homologs, and alleles of GPCRs. This identification can be made in vitro, e.g., under stringent hybridization conditions or PCR (e.g., using primers that hybridize to SEQ ID NO:1, SEQ ID NO:3, SEQ ID NO:5, SEQ ID NO:7, SEQ ID NO:9, SEQ ID NO:11, SEQ ID NO:13, SEQ ID NO:15, or SEQ ID NO:17) and sequencing, or by using the sequence information in a computer system for comparison with other nucleotide sequences. Typically, identification of polymorphic variants and alleles of a GPCR is made by comparing an amino acid sequence of about 25 amino acids or more, e.g., 50-100 amino acids. Amino acid identity of approximately at least 70% or above, optionally 75%, 80%, 85% or 90-95% or above typically demonstrates that a protein is a polymorphic variant, interspecies homolog, or allele of a GPCR. Sequence comparison is performed using the BLAST and BLAST 2.0 sequence comparison algorithms with default parameters, discussed below. Antibodies that bind specifically to a GPCR or a conserved region thereof can also be used to identify alleles, interspecies homologs, and polymorphic variants. The polymorphic variants, alleles and interspecies homologs are expected to retain the seven transmembrane structure of a G-protein coupled receptor.

GPCR nucleotide and amino acid sequence information may also be used to construct models of GPCRs in a computer system. These models are subsequently used to identify compounds that can activate or inhibit GPCRs. Such compounds that modulate the activity of a GPCR can be used, e.g., to investigate the role of GPCRs in signal transduction.

Definitions

“GPCR” and “TGR-GPCR” refer to novel G-protein coupled receptors. Particular GPCR of the current invention include “TGR342” (or “TGR-342”, “MCHR2” or “MCHR-2”); “TGR60”, “TGR346”, and “TGR339”. The GPCRs of the invention have seven transmembrane regions and have “G-protein coupled receptor activity,” e.g., they bind to G-proteins in response to extracellular stimuli and promote production of second messengers such as IP3, cAMP and Ca²⁺ via stimulation of downstream effectors such as phospholipase C and adenylate cyclase (for a description of the structure and function of GPCRs, see, e.g., Fong, supra, and Baldwin, supra).

Topologically, GPCRs have an N-terminal “extracellular domain,” a “transmembrane domain” comprising seven transmembrane regions and corresponding cytoplasmic and extracellular loops, and a C-terminal “cytoplasmic domain” (see, e.g., Buck & Axel, Cell 65:175-187 (1991)). These domains can be structurally identified using methods known to those of skill in the art, such as sequence analysis programs that identify hydrophobic and hydrophilic domains (see, e.g., Kyte & Doolittle, J. Mol. Biol. 157:105-132 (1982)). Such domains are useful for making chimeric proteins and for in vitro assays of the invention.

“Extracellular domain” therefore refers to the domain of a GPCR that protrudes from the cellular membrane and often binds to an extracellular ligand. This domain is often useful for in vitro ligand binding assays, both soluble and solid phase.

“Transmembrane domain,” comprises seven transmembrane regions plus the corresponding cytoplasmic and extracellular loops. Certain regions of the transmembrane domain can also be involved in ligand binding.

“Cytoplasmic domain” refers to the domain of a GPCR that protrudes into the cytoplasm after the seventh transmembrane region and continues to the C-terminus of the polypeptide.

“GPCR activity” refers to the ability of a GPCR to transduce a signal. Such activity can be measured, e.g., in a heterologous cell, by coupling a GPCR (or a chimeric GPCR) to a G-protein and a downstream effector such as PLC, and measuring increases in intracellular calcium (see, e.g., Offermans & Simon, J. Biol. Chem. 270:15175-15180 (1995)). Receptor activity can be effectively measured by recording ligand-induced changes in [Ca²⁺]ᵢ using fluorescent Ca²⁺-indicator dyes and fluorometric imaging.

The terms “GPCR” and “TGR-342, -60, -346, -and 399” therefore refer to polymorphic variants, alleles, mutants, and interspecies homologs and GPCR domains thereof that: (1) have about 70% amino acid sequence identity, preferably about 75, 80, 85, 90 or 95% or higher amino acid sequence identity, to SEQ ID NO:2; SEQ ID NO:4, SEQ ID NO:6, SEQ ID NO:8, SEQ ID NO:10, SEQ ID NO:12, SEQ ID NO:16, or SEQ ID NO:18 over a window of about 25 amino acids, preferably 50-100 amino acids; (2) bind to antibodies raised against an immunogen comprising an amino acid sequence of SEQ ID NO:2; SEQ ID NO:4, SEQ ID NO:6, SEQ ID NO:8, SEQ ID NO:10, SEQ ID NO:12, SEQ ID NO:16, or SEQ ID NO:18 and conservatively modified variants thereof; (3) specifically hybridize (with a size of at least about 100, preferably at least about 500 or 1000 nucleotides) under stringent hybridization conditions to a sequence SEQ ID NO:1; SEQ ID NO:3, SEQ ID NO:5, SEQ ID NO:7, SEQ ID NO:9, SEQ ID NO:11, SEQ ID NO:15, or SEQ ID NO:17, and conservatively modified variants thereof; or (4) have a nucleic acid sequence that has greater than about 95%, preferably greater than about 96%, 97%, 98%, 99%, or higher nucleotide sequence identity, preferably over a region of at least about 50, 100, 200, 500, 1000, or more nucleotides, to SEQ ID NO:1; SEQ ID NO:3, SEQ ID NO:5, SEQ ID NO:7, SEQ ID NO:9, SEQ ID NO:11, SEQ ID NO:15, or SEQ ID NO:17; (5) are amplified by primers that specifically hybridize under stringent conditions to SEQ ID NO: SEQ ID NO:1; SEQ ID NO:3, SEQ ID NO:5, SEQ ID NO:7, SEQ ID NO:9, SEQ ID NO:1, SEQ ID NO:15, or SEQ ID NO:17. This term also refers to a domain of a GPCR, as described above, or a fusion protein comprising a domain of a GPCR linked to a heterologous protein. A TGR-342, -60, -346, or -339 protein or domain typically comprises 10, 15, often 20, 25, or 30 or more contiguous amino acids of SEQ ID NO:2, 4, 6, 8, 10, or 12. A TGR-342, -60, -346, or TGR-339 nucleic acid typically comprises at least 15, often 20, 25, 30, or 50 or more contiguous nucleotides of a sequence of SEQ ID NOs: 1, 3, 5, 7, or 9. GPCR polynucleotide or polypeptide sequence of the invention is typically from a mammal including, but not limited to, human, rat, mouse, hamster, cow, pig, horse, sheep, or any mammal. A “TGR-342, -60, -346, and -339 polynucleotide” and a “TGR-342, -60, -346, and -339 polypeptide,” are both either naturally occurring or recombinant.

A “host cell” is a naturally occurring cell or a transformed cell that contains an expression vector and supports the replication or expression of the expression vector. Host cells may be cultured cells, explants, cells in vivo, and the like. Host cells may be prokaryotic cells such as E. coli, or eukaryotic cells such as yeast, insect, amphibian, or mammalian cells such as CHO, HeLa, and the like.

“Biological sample” as used herein is a sample of biological tissue or fluid that contains nucleic acids or polypeptides of novel GPCRs. Such samples include, but are not limited to, tissue isolated from humans, mice, and rats. Biological samples may also include sections of tissues such as frozen sections taken for histologic purposes. A biological sample is typically obtained from a eukaryotic organism, such as insects, protozoa, birds, fish, reptiles, and preferably a mammal such as rat, mouse, cow, dog, guinea pig, or rabbit, and most preferably a primate such as chimpanzees or humans. Preferred tissues typically depend on the known expression profile of the GPCR, and include e.g., normal colon, spleen, kidney, liver, hypothalamus, adipose, or other tissues.

The phrase “functional effects” in the context of assays for testing compounds that modulate GPCR-mediated signal transduction includes the determination of any parameter that is indirectly or directly under the influence of a GPCR, e.g., a functional, physical, or chemical effect. It includes ligand binding, changes in ion flux, membrane potential, current flow, transcription, G-protein binding, gene amplification, expression in cancer cells, GPCR phosphorylation or dephosphorylation, signal transduction, receptor-ligand interactions, second messenger concentrations (e.g., cAMP, cGMP, EP₃, or intracellular Ca²⁺), in vitro, in vivo, and ex vivo and also includes other physiologic effects such increases or decreases of neurotransmitter or hormone release.

By “determining the functional effect” is meant assays for a compound that increases or decreases a parameter that is indirectly or directly under the influence of a GPCR, e.g., functional, physical and chemical effects. Such functional effects can be measured by any means known to those skilled in the art, e.g., changes in spectroscopic characteristics (e.g., fluorescence, absorbance, refractive index), hydrodynamic (e.g., shape), chromatographic, or solubility properties, patch clamping, voltage-sensitive dyes, whole cell currents, radioisotope efflux, inducible markers, transcriptional activation of GPCRs; ligand binding assays; voltage, membrane potential and conductance changes; ion flux assays; changes in intracellular second messengers such as cAMP and inositol triphosphate (IP3); changes in intracellular calcium levels; neurotransmitter release, and the like.

“Inhibitors,” “activators,” and “modulators” of GPCRs are used interchangeably to refer to inhibitory, activating, or modulating molecules identified using in vitro and in vivo assays for signal transduction, e.g., ligands, agonists, antagonists, and their homologs and mimetics. Inhibitors are compounds that, e.g., bind to, partially or totally block stimulation, decrease, prevent, delay activation, inactivate, desensitize, or down regulate signal transduction, e.g., antagonists. Activators are compounds that, e.g., bind to, stimulate, increase, open, activate, facilitate, enhance activation, sensitize or up regulate signal transduction, e.g., agonists. Modulators include compounds that, e.g., alter the interaction of a polypeptide with: extracellular proteins that bind activators or inhibitors; G-proteins; G-protein alpha, beta, and gamma subunits; and kinases. Modulators also include genetically modified versions of GPCRs, e.g., with altered activity, as well as naturally occurring and synthetic ligands, antagonists, agonists, antibodies, small chemical molecules and the like. Such assays for inhibitors and activators include, e.g., expressing GPCRs in vitro, in cells, or cell membranes, applying putative modulator compounds, and then determining the functional effects on signal transduction, as described above.

Samples or assays comprising GPCRs that are treated with a potential activator, inhibitor, or modulator are compared to control samples without the inhibitor, activator, or modulator to examine the extent of inhibition. Control samples (untreated with inhibitors) are assigned a relative GPCR activity value of 100%. Inhibition of a GPCR is achieved when the GPCR activity value relative to the control is about 80%, preferably 50%, more preferably 25-0%. Activation of a GPCR is achieved when the GPCR activity value relative to the control (untreated with activators) is 110%, more preferably 150%, more preferably 200-500% (i.e., two to five fold higher relative to the control), more preferably 1000-3000% higher.

The terms “isolated” “purified” or “biologically pure” refer to material that is substantially or essentially free from components which normally accompany it as found in its native state. Purity and homogeneity are typically determined using analytical chemistry techniques such as polyacrylamide gel electrophoresis or high performance liquid chromatography. A protein that is the predominant species present in a preparation is substantially purified. In particular, an isolated GPCR nucleic acid is separated from open reading frames that flank the GPCR gene and encode proteins other than the GPCR. The term “purified” denotes that a nucleic acid or protein gives rise to essentially one band in an electrophoretic gel. Particularly, it means that the nucleic acid or protein is at least 85% pure, more preferably at least 95% pure, and most preferably at least 99% pure.

“Biologically active” GPCR refers to a GPCR having signal transduction activity and G protein coupled receptor activity, as described above.

“Nucleic acid” refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, mRNA, oligonucleotide, and polynucleotide.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. The term “amino acid analogs” refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. The term “amino acid mimetics” refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M) (see, e.g., Creighton, Proteins (1984)).

Macromolecular structures such as polypeptide structures can be described in terms of various levels of organization. For a general discussion of this organization, see, e.g., Alberts et al., Molecular Biology of the Cell (3^{rd} ed., 1994) and Cantor and Schimmel, Biophysical Chemistry Part I: The Conformation of Biological Macromolecules (1980). “Primary structure” refers to the amino acid sequence of a particular peptide. “Secondary structure” refers to locally ordered, three dimensional structures within a polypeptide. These structures are commonly known as domains. Domains are portions of a polypeptide that form a compact unit of the polypeptide and are typically 25 to approximately 500 amino acids long. Typical domains are made up of sections of lesser organization such as stretches of β-sheet and α-helices. “Tertiary structure” refers to the complete three dimensional structure of a polypeptide monomer. “Quaternary structure” refers to the three dimensional structure formed by the noncovalent association of independent tertiary units. Anisotropic terms are also known as energy terms.

A “label” or a “detectable moiety” is a composition detectable by spectroscopic, photochemical, biochemical, immunochemical, or chemical means. For example, useful labels include ³²P, fluorescent dyes, electron-dense reagents, enzymes (e.g., as commonly used in an ELISA), biotin, digoxigenin, or haptens and proteins which can be made detectable (e.g., by incorporating a radiolabel into the peptide, and used to detect antibodies specifically reactive with the peptide).

A “labeled nucleic acid probe or oligonucleotide” is one that is bound, either covalently, through a linker or a chemical bond, or noncovalently, through ionic, van der Waals, electrostatic, or hydrogen bonds to a label such that the presence of the probe may be detected by detecting the presence of the label bound to the probe.

As used herein a “nucleic acid probe or oligpnucleotide” is defined as a nucleic acid capable of binding to a target nucleic acid of complementary sequence through one or more types of chemical bonds, usually through complementary base pairing, usually through hydrogen bond formation. As used herein, a probe may include natural (i.e., A, G, C, or T) or modified bases (7-deazaguanosine, inosine, etc.). In addition, the bases in a probe may be joined by a linkage other than a phosphodiester bond, so long as it does not interfere with hybridization. Thus, for example, probes may be peptide nucleic acids in which the constituent bases are joined by peptide bonds rather than phosphodiester linkages. It will be understood by one of skill in the art that probes may bind target sequences lacking complete complementarity with the probe sequence depending upon the stringency of the hybridization conditions. The probes are preferably directly labeled as with isotopes, chromophores, lumiphores, chromogens, or indirectly labeled such as with biotin to which a streptavidin complex may later bind. By assaying for the presence or absence of the probe, one can detect the presence or absence of the select sequence or subsequence.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

A “promoter” is defined as an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. A “constitutive” promoter is a promoter that is active under most environmental and developmental conditions. An “inducible” promoter is a promoter that is active under environmental or developmental regulation. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

An “expression vector” is a nucleic acid construct, generated recombinantly or synthetically, with a series of specified nucleic acid elements that permit transcription of a particular nucleic acid in a host cell. The expression vector can be part of a plasmid, virus, or nucleic acid fragment. Typically, the expression vector includes a nucleic acid to be transcribed operably linked to a promoter.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 70% identity, preferably 75%, 80%, 85%, 90%, or 95% identity over a specified region, when compared and aligned for maximum correspondence over a comparison window, or designated region as measured using a BLAST or BLAST 2.0 sequence comparison algorithms with default parameters described below, or by manual alignment and visual inspection. Such sequences are then said to be “substantially identical.” This definition also refers to the compliment of a test sequence. Preferably, the identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and, TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

A preferred example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The phrase “selectively (or specifically) hybridizes to” refers to the binding, duplexing, or hybridizing of a molecule only to a particular nucleotide sequence under stringent hybridization conditions when that sequence is present in a complex mixture (e.g., total cellular or library DNA or RNA).

The phrase “stringent hybridization conditions” refers to conditions under which a probe will hybridize to its target subsequence, typically in a complex mixture of nucleic acid, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency.

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA-methodologies (e.g., single chain Fv) or those identified using phage display libraries (see, e.g., McCafferty et al., Nature 348:552-554 (1990)).

For preparation of monoclonal or polyclonal antibodies, any technique known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy (1985)). Techniques for the production of single chain antibodies (U.S. Pat. No. 4,946,778) can be adapted to produce antibodies to polypeptides of this invention. Also, transgenic mice, or other organisms such as other mammals, may be used to express humanized antibodies. Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)).

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

An “anti-GPCR” antibody is an antibody or antibody fragment that specifically binds a polypeptide encoded by a GPCR gene, cDNA, or a subsequence thereof.

The term “immunoassay” is an assay that uses an antibody to specifically bind an antigen. The immunoassay is characterized by the use of specific binding properties of a particular antibody to isolate, target, and/or quantify the antigen.

The phrase “specifically (or selectively) binds” to an antibody or “specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein. For example, polyclonal antibodies raised to a particular GPCR can be selected to obtain only those polyclonal antibodies that are specifically immunoreactive with the GPCR, and not with other proteins, except for polymorphic variants, orthologs, and alleles of the GPCR. This selection may be achieved by subtracting out antibodies that cross-react with GPCR molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity). Typically a specific or selective reaction will be at least twice background signal or noise and more typically more than 10 to 100 times background. Antibodies that react only with a particular GPCR ortholog, e.g., from specific species such as rat, mouse, or human, can also be made as described above, by subtracting out antibodies that bind to the same GPCR from another species.

The phrase “selectively associates with” refers to the ability of a nucleic acid to “selectively hybridize” with another as defined above, or the ability of an antibody to selectively (or specifically) bind to a protein, as defined above.

Isolation of Nucleic Acids Encoding GPCRs

A. General Recombinant DNA Methods

This invention relies on routine techniques in the field of recombinant genetics. Basic texts disclosing the general methods of use in this invention include Sambrook et al., Molecular Cloning, A Laboratory Manual (2nd ed. 1989); Kriegler, Gene Transfer and Expression: A Laboratory Manual (1990); and Current Protocols in Molecular Biology (Ausubel et al., eds., 1994)).

For nucleic acids, sizes are given in either kilobases (kb) or base pairs (bp). These are estimates derived from agarose or acrylamide gel electrophoresis, from sequenced nucleic acids, or from published DNA sequences. For proteins, sizes are given in kilodaltons (kDa) or amino acid residue numbers. Proteins sizes are estimated from gel electrophoresis, from sequenced proteins, from derived amino acid sequences, or from published protein sequences.

Oligonucleotides that are not commercially available can be chemically synthesized according to the solid phase phosphoramidite triester method first described by Beaucage & Caruthers, Tetrahedron Letts. 22:1859-1862 (1981), using an automated synthesizer, as described in Van Devanter et. al., Nucleic Acids Res. 12:6159-6168 (1984). Purification of oligonucleotides is by either native acrylamide gel electrophoresis or by anion-exchange HPLC as described in Pearson & Reanier, J. Chrom. 255:137-149 (1983).

The sequence of the cloned genes and synthetic oligonucleotides can be verified after cloning using, e.g., the chain termination method for sequencing double-stranded templates of Wallace et al., Gene 16:21-26 (1981).

B. Cloning Methods for the Isolation of Nucleotide Sequences Encoding GPCRs

In general, the nucleic acid sequences encoding GPCRs and related nucleic acid sequence homologs are cloned from cDNA and genomic DNA libraries by hybridization with a probe, or isolated using amplification techniques with oligonucleotide primers. For example, GPCR sequences are typically isolated from mammalian nucleic acid (genomic or cDNA) libraries by hybridizing with a nucleic acid probe, the sequence of which can be derived from SEQ ID NO:1; SEQ ID NO:3, SEQ ID NO:5, SEQ ID NO:7, SEQ ID NO:9, SEQ ID NO:11, SEQ ID NO:15, or SEQ ID NO:17. Suitable tissues from which GPCR RNA and cDNA can be isolated include, e.g., liver, kidney, hypothalamus, spleen, colon, adipose, and other tissues.

Amplification techniques using primers can also be used to amplify and isolate GPCR nucleic acids from DNA or RNA. Examples of suitable primers for amplification of specific GPCRs include those set forth in the Example Section (see, e.g., Dieffenfach & Dveksler, PCR Primer: A Laboratory Manual (1995)). These primers can be used, e.g., to amplify either the full length sequence or a probe of one to several hundred nucleotides, which is then used to screen a mammalian library for full-length GPCRs.

Nucleic acids encoding GPCRs can also be isolated from expression libraries using antibodies as probes. Such polyclonal or monoclonal antibodies can be raised using the sequence of SEQ ID NO:2; SEQ ID NO:4, SEQ ID NO:6, SEQ ID NO:8, SEQ ID NO:10, SEQ ID NO:112, SEQ ID NO:16, or SEQ ID NO:18.

GPCR polymorphic variants, alleles, and interspecies homologs that are substantially identical to a GPCR can be isolated using GPCR nucleic acid probes, and oligonucleotides under stringent hybridization conditions, by screening libraries. Alternatively, expression libraries can be used to clone GPCRs and GPCR polymorphic variants, alleles, and interspecies homologs, by detecting expressed homologs immunologically with antisera or purified antibodies made against GPCRs, which also recognize and selectively bind to the GPCR homolog.

To make a cDNA library, one should choose a source that is rich in GPCR mRNA. The mRNA is then made into cDNA using reverse transcriptase, ligated into a recombinant vector, and transfected into a recombinant host for propagation, screening and cloning. Methods for making and screening cDNA libraries are well known (see, e.g., Gubler & Hoffman, Gene 25:263-269 (1983); Sambrook et al., supra; Ausubel et al., supra).

For a genomic library, the DNA is extracted from the tissue and either mechanically sheared or enzymatically digested to yield fragments of about 12-20 kb. The fragments are then separated by gradient centrifugation from undesired sizes and are constructed in bacteriophage lambda vectors. These vectors and phage are packaged in vitro. Recombinant phage are analyzed by plaque hybridization as described in Benton & Davis, Science 196:180-182 (1977). Colony hybridization is carried out as generally described in Grunstein et al., Proc. Natl. Acad. Sci. USA., 72:3961-3965 (1975).

An alternative method of isolating GPCR nucleic acids and their homologs combines the use of synthetic oligonucleotide primers and amplification of an RNA or DNA template (see U.S. Pat. Nos. 4,683,195 and 4,683,202; PCR Protocols: A Guide to Methods and Applications (Innis et al., eds, 1990)). Methods such as polymerase chain reaction (PCR) and ligase chain reaction (LCR) can be used to amplify nucleic acid sequences of GPCRs directly from mRNA, from cDNA, from genomic libraries or cDNA libraries. Degenerate oligonucleotides can be designed to amplify GPCR homologs using the sequences provided herein. Restriction endonuclease sites can be incorporated into the primers. Polymerase chain reaction or other in vitro amplification methods may also be useful, for example, to clone nucleic acid sequences that code for proteins to be expressed, to make nucleic acids to use as probes for detecting the presence of GPCR-encoding mRNA in physiological samples, for nucleic acid sequencing, or for other purposes. Genes amplified by the PCR reaction can be purified from agarose gels and cloned into an appropriate vector.

Gene expression of GPCRs can also be analyzed by techniques known in the art, e.g., reverse transcription and amplification of mRNA, isolation of total RNA or poly A⁺ RNA, northern blotting, dot blotting, in situ hybridization, RNase protection, probing DNA microchip arrays, and the like. In one embodiment, high density oligonucleotide analysis technology (e.g., GeneChip™) is used to identify homologs and polymorphic variants of the GPCRs of the invention. In the case where the homologs being identified are linked to a known disease, they can be used with GeneChip™ as a diagnostic tool in detecting the disease in a biological sample, see, e.g., Gunthand et al., AIDS Res. Hum. Retroviruses 14: 869-876 (1998); Kozal et al., Nat. Med. 2:753-759 (1996); Matson et al., Anal. Biochem. 224:110-106 (1995); Lockhart et al., Nat. Biotechnol. 14:1675-1680 (1996); Gingeras et al., Genome Res. 8:435-448 (1998); Hacia et al., Nucleic Acids Res. 26:3865-3866 (1998).

Synthetic oligonucleotides can be used to construct recombinant GPCR genes for use as probes or for expression of protein. This method is performed using a series of overlapping oligonucleotides usually 40-120 bp in length, representing both the sense and nonsense strands of the gene. These DNA fragments are then annealed, ligated and cloned. Alternatively, amplification techniques can be used with precise primers to amplify a specific subsequence of the GPCR nucleic acid. The specific subsequence is then ligated into an expression vector.

The nucleic acid encoding a GPCR is typically cloned into intermediate vectors before transformation into prokaryotic or eukaryotic cells for replication and/or expression. These intermediate vectors are typically prokaryote vectors, e.g., plasmids, or shuttle vectors.

Optionally, nucleic acids encoding chimeric proteins comprising GPCRs or domains thereof can be made according to standard techniques. For example, a domain such as ligand binding domain, an extracellular domain, a transmembrane domain (e.g., one comprising seven transmembrane regions and corresponding extracellular and cytosolic loops), the transmembrane domain and a cytoplasmic domain, an active site, a subunit association region, etc., can be covalently linked to a heterologous protein. For example, an extracellular domain can be linked to a heterologous GPCR transmembrane domain, or a heterologous GPCR extracellular domain can be linked to a transmembrane domain. Other heterologous proteins of choice include, e.g., green fluorescent protein, luciferase, or β-gal.

C. Expression in Prokaryotes and Eukaryotes

To obtain high level expression of a cloned gene or nucleic acid, such as those cDNAs encoding GPCRs, one typically subclones a GPCR into an expression vector that contains a strong promoter to direct transcription, a transcription/translation terminator, and if for a nucleic acid encoding a protein, a ribosome binding site for translational initiation. Suitable bacterial promoters are well known in the art and described, e.g., in Sambrook et al. and Ausubel et al. Bacterial expression systems for expressing the GPCR protein are available in, e.g., E. coli, Bacillus sp., and Salmonella (Palva et al., Gene 22:229-235 (1983); Mosbach et al., Nature 302:543-545 (1983). Kits for such expression systems are commercially available. Eukaryotic expression systems for mammalian cells, yeast, and insect cells are well known in the art and are also commercially available. In one embodiment, the eukaryotic expression vector is an adenoviral vector, an adeno-associated vector, or a retroviral vector.

The promoter used to direct expression of a heterologous nucleic acid depends on the particular application. The promoter is optionally positioned about the same distance from the heterologous transcription start site as it is from the transcription start site in its natural setting. As is known in the art, however, some variation in this distance can be accommodated without loss of promoter function.

In addition to the promoter, the expression vector typically contains a transcription unit or expression cassette that contains all the additional elements required for the expression of the GPCR encoding nucleic acid in host cells. A typical expression cassette thus contains a promoter operably linked to the nucleic acid sequence encoding a GPCR and signals required for efficient polyadenylation of the transcript, ribosome binding sites, and translation termination. The nucleic acid sequence encoding a GPCR may typically be linked to a cleavable signal peptide sequence to promote secretion of the encoded protein by the transformed cell. Such signal peptides would include, among others, the signal peptides from tissue plasminogen activator, insulin, and neuron growth factor, and juvenile hormone esterase of Heliothis virescens. Additional elements of the cassette may include enhancers and, if genomic DNA is used as the structural gene, introns with functional splice donor and acceptor sites.

In addition to a promoter sequence, the expression cassette should also contain a transcription termination region downstream of the structural gene to provide for efficient termination. The termination region may be obtained from the same gene as the promoter sequence or may be obtained from different genes.

The particular expression vector used to transport the genetic information into the cell is not particularly critical. Any of the conventional vectors used for expression in eukaryotic or prokaryotic cells may be used. Standard bacterial expression vectors include plasmids such as pBR322 based plasmids, pSKF, pET23D, and fusion expression systems such as GST and LacZ. Epitope tags can also be added to recombinant proteins to provide convenient methods of isolation, e.g., c-myc.

Expression vectors containing regulatory elements from eukaryotic viruses are typically used in eukaryotic expression vectors, e.g., SV40 vectors, papilloma virus vectors, and vectors derived from Epstein-Barr virus. Other exemplary eukaryotic vectors include pMSG, pAV009/A⁺, pMTO10/A⁺, pMAMneo-5, baculovirus pDSVE, and any other vector allowing expression of proteins under the direction of the SV40 early promoter, SV40 later promoter, metallothionein promoter, murine mammary tumor virus promoter, Rous sarcoma virus promoter, polyhedrin promoter, or other promoters shown effective for expression in eukaryotic cells.

Some expression systems have markers that provide gene amplification such as thymidine kinase, hygromycin B phosphotransferase, and dihydrofolate reductase. Alternatively, high yield expression systems not involving gene amplification are also suitable, such as using a baculovirus vector in insect cells, with a GPCR-encoding sequence under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable. The prokaryotic sequences are optionally chosen such that they do not interfere with the replication of the DNA in eukaryotic cells, if necessary.

Standard transfection methods are used to produce bacterial, mammalian, yeast or insect cell lines that express large quantities of GPCR protein, which are then purified using standard techniques (see, e.g., Colley et al., J. Biol. Chem. 264:17619-17622 (1989); Guide to Protein Purification, in Methods in Enzymology, vol. 182 (Deutscher, ed., 1990)). Transformation of eukaryotic and prokaryotic cells are performed according to standard techniques (see, e.g., Morrison, J. Bact. 132:349-351 (1977); Clark-Curtiss & Curtiss, Methods in Enzymology 101:347-362 (Wu et al., eds, 1983).

Any of the well known procedures for introducing foreign nucleotide sequences into host cells may be used. These include the use of calcium phosphate transfection, polybrene, protoplast fusion, electroporation, liposomes, microinjection, plasma vectors, viral vectors and any of the other well known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing a GPCR.

After the expression vector is introduced into the cells, the transfected cells are cultured under conditions favoring expression of a GPCR, which is recovered from the culture using standard techniques identified below.

Purification of GPCRs

Either naturally occurring or recombinant GPCRs can be purified for use in functional assays. Optionally, recombinant GPCRs are purified. Naturally occurring GPCRs are purified, e.g., from any suitable tissue or cell expressing naturally occurring GPCRs. Recombinant GPCRs are purified from any suitable bacterial or eukaryotic expression system, e.g., CHO cells or insect cells.

A GPCR may be purified to substantial purity by standard techniques, including selective precipitation with such substances as ammonium sulfate; column chromatography, immunopurification methods, and others (see, e.g., Scopes, Protein Purification: Principles and Practice (1982); U.S. Pat. No. 4,673,641; Ausubel et al., supra; and Sambrook et al., supra).

A number of procedures can be employed when a recombinant GPCR is being purified. For example, proteins having established molecular adhesion properties can be reversibly fused to a GPCR. With the appropriate ligand, a GPCR can be selectively adsorbed to a purification column and then freed from the column in a relatively pure form. The fused protein is then removed by enzymatic activity. Finally, a GPCR could be purified using immunoaffinity columns.

A. Purification of GPCRs from Recombinant Cells

Recombinant proteins are expressed by transformed bacteria or eukaryotic cells such as CHO cells or insect cells in large amounts, typically after promoter induction; but expression can be constitutive. Promoter induction with IPTG is a one example of an inducible promoter system. Cells are grown according to standard procedures in the art. Fresh or frozen cells are used for isolation of protein.

Proteins expressed in bacteria may form insoluble aggregates (“inclusion bodies”). Several protocols are suitable for purification of GPCR inclusion bodies. For example, purification of inclusion bodies typically involves the extraction, separation and/or purification of inclusion bodies by disruption of bacterial cells, e.g., by incubation in a buffer of 50 mM TRIS/HCL pH 7.5, 50 mM NaCl, 5 mM MgCl₂, 1 mM DTT, 0.1 mM ATP, and 1 mM PMSF. The cell suspension can be lysed using 2-3 passages through a French Press, homogenized using a Polytron (Brinkman Instruments) or sonicated on ice. Alternate methods of lysing bacteria are apparent to those of skill in the art (see, e.g., Sambrook et al., supra; Ausubel et al., supra).

If necessary, the inclusion bodies are solubilized, and the lysed cell suspension is typically centrifuged to remove unwanted insoluble matter. Proteins that formed the inclusion bodies may be renatured by dilution or dialysis with a compatible buffer. Suitable solvents include, but are not limited to urea (from about 4 M to about 8 M), formamide (at least about 80%, volume/volume basis), and guanidine hydrochloride (from about 4 M to about 8 M). Some solvents which are capable of solubilizing aggregate-forming proteins, for example SDS (sodium dodecyl sulfate), 70% formic acid, are inappropriate for use in this procedure due to the possibility of irreversible denaturation of the proteins, accompanied by a lack of immunogenicity and/or activity. Although guanidine hydrochloride and similar agents are denaturants, this denaturation is not irreversible and renaturation may occur upon removal (by dialysis, for example) or dilution of the denaturant, allowing re-formation of immunologically and/or biologically active protein. Other suitable buffers are known to those skilled in the art. The GPCR is separated from other bacterial proteins by standard separation techniques, e.g., with Ni—NTA agarose resin.

Alternatively, it is possible to purify the GPCR from bacteria periplasm. After lysis of the bacteria, when the GPCR is exported into the periplasm of the bacteria, the periplasmic fraction of the bacteria can be isolated by cold osmotic shock in addition to other methods known to skill in the art. To isolate recombinant proteins from the periplasm, the bacterial cells are centrifuged to form a pellet. The pellet is resuspended in a buffer containing 20% sucrose. To lyse the cells, the bacteria are centrifuged and the pellet is resuspended in ice-cold 5 mM MgSO₄ and kept in an ice bath for approximately 10 minutes. The cell suspension is centrifuged and the supernatant decanted and saved. The recombinant proteins present in the supernatant can be separated from the host proteins by standard separation techniques well known to those of skill in the art.

B. Standard Protein Separation Techniques for Purifying GPCRs Solubility Fractionation

Often as an initial step, particularly if the protein mixture is complex, an initial salt fractionation can separate many of the unwanted host cell proteins (or proteins derived from the cell culture media) from the recombinant protein of interest. The preferred salt is ammonium sulfate. Ammonium sulfate precipitates proteins by effectively reducing the amount of water in the protein mixture. Proteins then precipitate on the basis of their solubility. The more hydrophobic a protein is, the more likely it is to precipitate at lower ammonium sulfate concentrations. A typical protocol includes adding saturated ammonium sulfate to a protein solution so that the resultant ammonium sulfate concentration is between 20-30%. This concentration will precipitate the most hydrophobic of proteins. The precipitate is then discarded (unless the protein of interest is hydrophobic) and ammonium sulfate is added to the supernatant to a concentration known to precipitate the protein of interest. The precipitate is then solubilized in buffer and the excess salt removed if necessary, either through dialysis or diafiltration. Other methods that rely on solubility of proteins, such as cold ethanol precipitation, are well known to those of skill in the art and can be used to fractionate complex protein mixtures.

Size Differential Filtration

The molecular weight of a GPCR can be used to isolate it from proteins of greater and lesser size using ultrafiltration through membranes of different pore size (for example, Amicon or Millipore membranes). As a first step, the protein mixture is ultrafiltered through a membrane with a pore size that has a lower molecular weight cut-off than the molecular weight of the protein of interest. The retentate of the ultrafiltration is then ultrafiltered against a membrane with a molecular cut off greater than the molecular weight of the protein of interest. The recombinant protein will pass through the membrane into the filtrate. The filtrate can then be chromatographed as described below.

Column Chromatography

GPCRs can also be separated from other proteins on the basis of size, net surface charge, hydrophobicity, and affinity for ligands. In addition, antibodies raised against proteins can be conjugated to column matrices and the proteins immunopurified. All of these methods are well known in the art. It will be apparent to one of skill that chromatographic techniques can be performed at any scale and using equipment from many different manufacturers (e.g., Pharmacia Biotech).

Immunological Detection of GPCRs

In addition to the detection of GPCR genes and gene expression using nucleic acid hybridization technology, one can also use immunoassays to detect GPCRs, e.g., to identify cells such as kidney cells, liver cells, adipocytes, hypothalamus cells, spleen cells, or colon cells, and variants of GPCRs. Immunoassays can be used to qualitatively or quantitatively analyze GPCRs. A general overview of the applicable technology can be found in Harlow & Lane, Antibodies: A Laboratory Manual (1988).

A. Antibodies to GPCRs

Methods of producing polyclonal and monoclonal antibodies that react specifically with GPCRs are known to those of skill in the art (see, e.g., Coligan, Current Protocols in Immunology (1991); Harlow & Lane, supra; Goding, Monoclonal Antibodies: Principles and Practice (2d ed. 1986); and Kohler & Milstein, Nature 256:495-497 (1975). Such techniques include antibody preparation by selection of antibodies from libraries of recombinant antibodies in phage or similar vectors, as well as preparation of polyclonal and monoclonal antibodies by immunizing rabbits or mice (see, e.g., Huse et al., Science 246:1275-1281 (1989); Ward et al., Nature 341:544-546 (1989)). Such antibodies can be used for therapeutic and diagnostic applications, e.g., in the treatment and/or detection of any of the GPCR-associated diseases or conditions described herein.

A number of GPCRs comprising immunogens may be used to produce antibodies specifically reactive with GPCRs. For example, a recombinant GPCR or an antigenic fragment thereof, is isolated as described herein. Recombinant protein can be expressed in eukaryotic or prokaryotic cells as described above, and purified as generally described above. Recombinant protein is the preferred immunogen for the production of monoclonal or polyclonal antibodies. Alternatively, a synthetic peptide derived from the sequences disclosed herein and conjugated to a carrier protein can be used an immunogen. Naturally occurring protein may also be used either in pure or impure form. The product is then injected into an animal capable of producing antibodies. Either monoclonal or polyclonal antibodies may be generated, for subsequent use in immunoassays to measure the protein.

Methods of production of polyclonal antibodies are known to those of skill in the art. An inbred strain of mice (e.g., BALB/C mice) or rabbits is immunized with the protein using a standard adjuvant, such as Freund's adjuvant, and a standard immunization protocol. The animal's immune response to the immunogen preparation is monitored by taking test bleeds and determining the titer of reactivity to the GPCR. When appropriately high titers of antibody to the immunogen are obtained, blood is collected from the animal and antisera are prepared. Further fractionation of the antisera to enrich for antibodies reactive to the protein can be done if desired (see Harlow & Lane, supra).

Monoclonal antibodies may be obtained by various techniques familiar to those skilled in the art. Briefly, spleen cells from an animal immunized with a desired antigen are immortalized, commonly by fusion with a myeloma cell (see Kohler & Milstein, Eur. J. Immunol. 6:511-519 (1976)). Alternative methods of immortalization include transformation with Epstein Barr Virus, oncogenes, or retroviruses, or other methods well known in the art. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences which encode a monoclonal antibody or a binding fragment thereof by screening a DNA library from human B cells according to the general protocol outlined by Huse et al., Science 246:1275-1281 (1989).

Monoclonal antibodies and polyclonal sera are collected and titered against the immunogen protein in an immunoassay, for example, a solid phase immunoassay with the immunogen immobilized on a solid support. Typically, polyclonal antisera with a titer of 10⁴ or greater are selected and tested for their cross reactivity against non-GPCR proteins or even other related proteins from other organisms, using a competitive binding immunoassay. Specific polyclonal antisera and monoclonal antibodies will usually bind with a K_{d} of at least about 0.1 mM, more usually at least about 1 μM, optionally at least about 0.1 μM or better, and optionally 0.01 μM or better.

Once GPCR-specific antibodies are available, GPCRs can be detected by a variety of immunoassay methods. For a review of immunological and immunoassay procedures, see Basic and Clinical Immunology (Stites & Terr eds., 7th ed. 1991). Moreover, the immunoassays of the present invention can be performed in any of several configurations, which are reviewed extensively in Enzyme Immunoassay (Maggio, ed., 1980); and Harlow & Lane, supra.

B. Immunological Binding Assays

GPCRs can be detected and/or quantified using any of a number of well recognized immunological binding assays (see, e.g., U.S. Pat. Nos. 4,366,241; 4,376,110; 4,517,288; and 4,837,168). For a review of the general immunoassays, see also Methods in Cell Biology: Antibodies in Cell Biology, volume 37 (Asai, ed. 1993); Basic and Clinical Immunology (Stites & Terr, eds., 7th ed. 1991). Immunological binding assays (or immunoassays) typically use an antibody that specifically binds to a protein or antigen of choice (in this case the GPCR or antigenic subsequence thereof). The antibody (e.g., anti-GPCR) may be produced by any of a number of means well known to those of skill in the art and as described above.

Immunoassays also often use a labeling agent to specifically bind to and label the complex formed by the antibody and antigen. The labeling agent may itself be one of the moieties comprising the antibody/antigen complex. Thus, the labeling agent may be a labeled GPCR polypeptide or a labeled anti-GPCR antibody. Alternatively, the labeling agent may be a third moiety, such a secondary antibody, that specifically binds to the antibody/GPCR complex (a secondary antibody is typically specific to antibodies of the species from which the first antibody is derived). Other proteins capable of specifically binding immunoglobulin constant regions, such as protein A or protein G may also be used as the label agent. These proteins exhibit a strong non-immunogenic reactivity with immunoglobulin constant regions from a variety of species (see, e.g., Kronval et al., J. Immunol. 111: 1401-1406 (1973); Akerstrom et al., J. Immunol. 135:2589-2542 (1985)). The labeling agent can be modified with a detectable moiety, such as biotin, to which another molecule can specifically bind, such as streptavidin. A variety of detectable moieties are well known to those skilled in the art.

Throughout the assays, incubation and/or washing steps may be required after each combination of reagents. Incubation steps can vary from about 5 seconds to several hours, optionally from about 5 minutes to about 24 hours. However, the incubation time will depend upon the assay format, antigen, volume of solution, concentrations, and the like. Usually, the assays will be carried out at ambient temperature, although they can be conducted over a range of temperatures, such as 10° C. to 40° C.

Non-Competitive Assay Formats

Immunoassays for detecting GPCRs in samples may be either competitive or noncompetitive. Noncompetitive immunoassays are assays in which the amount of antigen is directly measured. In one preferred “sandwich” assay, for example, the anti-GPCR antibodies can be bound directly to a solid substrate on which they are immobilized. These immobilized antibodies then capture GPCRs present in the test sample. The GPCR is thus immobilized is then bound by a labeling agent, such as a second GPCR antibody bearing a label. Alternatively, the second antibody may lack a label, but it may, in turn, be bound by a labeled third antibody specific to antibodies of the species from which the second antibody is derived. The second or third antibody is typically modified with a detectable moiety, such as biotin, to which another molecule specifically binds, e.g., streptavidin, to provide a detectable moiety.

Competitive Assay Formats

In competitive assays, the amount of GPCR present in the sample is measured indirectly by measuring the amount of a known, added (exogenous) GPCR displaced (competed away) from an anti-GPCR antibody by the unknown GPCR present in a sample. In one competitive assay, a known amount of GPCR is added to a sample and the sample is then contacted with an antibody that specifically binds to the GPCR. The amount of exogenous GPCR bound to the antibody is inversely proportional to the concentration of GPCR present in the sample. In a particularly preferred embodiment, the antibody is immobilized on a solid substrate. The amount of GPCR bound to the antibody may be determined either by measuring the amount of GPCR present in a GPCR/antibody complex, or alternatively by measuring the amount of remaining uncomplexed protein. The amount of GPCR may be detected by providing a labeled GPCR molecule.

A hapten inhibition assay is another preferred competitive assay. In this assay the known GPCR, is immobilized on a solid substrate. A known amount of anti-GPCR antibody is added to the sample, and the sample is then contacted with the immobilized GPCR. The amount of anti-GPCR antibody bound to the known immobilized GPCR is inversely proportional to the amount of GPCR present in the sample. Again, the amount of immobilized antibody may be detected by detecting either the immobilized fraction of antibody or the fraction of the antibody that remains in solution. Detection may be direct where the antibody is labeled or indirect by the subsequent addition of a labeled moiety that specifically binds to the antibody as described above.

Cross-Reactivity Determinations

Immunoassays in the competitive binding format can also be used for crossreactivity determinations. For example, a protein at least partially encoded by SEQ ID NO:1; SEQ ID NO:3, SEQ ID NO:5, SEQ ID NO:7, SEQ ID NO:9, SEQ ID NO:11, SEQ ID NO:15, or SEQ ID NO:17 can be immobilized to a solid support. Proteins (e.g., GPCR proteins and homologs) are added to the assay that compete for binding of the antisera to the immobilized antigen. The ability of the added proteins to compete for binding of the antisera to the immobilized protein is compared to the ability of GPCRs encoded by SEQ ID NO:1; SEQ ID NO:3, SEQ ID NO:5, SEQ ID NO:7, SEQ ID NO:9, SEQ ID NO:11, SEQ ID NO:15, or SEQ ID NO:17 to compete with itself. The percent crossreactivity for the above proteins is calculated, using standard calculations. Those antisera with less than 10% cross-reactivity with each of the added proteins listed above are selected and pooled. The cross-reacting antibodies are optionally removed from the pooled antisera by immunoabsorption with the added considered proteins, e.g., distantly related homologs.

The immunoabsorbed and pooled antisera are then used in a competitive binding immunoassay as described above to compare a second protein, thought to be perhaps an allele or polymorphic variant of a GPCR, to the immunogen protein (i.e., the GPCR of SEQ ID NO:2; SEQ ID NO:4, SEQ ID NO:6, SEQ ID NO:8, SEQ ID NO:10, SEQ ID NO:12, SEQ ID NO:16, or SEQ ID NO:18). In order to make this comparison, the two proteins are each assayed at a wide range of concentrations and the amount of each protein required to inhibit 50% of the binding of the antisera to the immobilized protein is determined. If the amount of the second protein required to inhibit 50% of binding is less than 10 times the amount of the protein encoded by SEQ ID NO:1; SEQ ID NO:3, SEQ ID NO:5, SEQ ID NO:7, SEQ ID NO:9, SEQ ID NO:11, SEQ ID NO:15, or SEQ ID NO:17 that is required to inhibit 50% of binding, then the second protein is said to specifically bind to the polyclonal antibodies generated to a GPCR immunogen.

Other Assay Formats

Western blot (immunoblot) analysis is used to detect and quantify the presence of GPCR in the sample. The technique generally comprises separating sample proteins by gel electrophoresis on the basis of molecular weight, transferring the separated proteins to a suitable solid support, (such as a nitrocellulose filter, a nylon-filter, or derivatized nylon filter), and incubating the sample with the antibodies that specifically bind GPCR. The anti-GPCR antibodies specifically bind to the GPCR on the solid support. These antibodies may be directly labeled or alternatively may be subsequently detected using labeled antibodies (e.g., labeled sheep anti-mouse antibodies) that specifically bind to the anti-GPCR antibodies.

Other assay formats include liposome immunoassays (LIA), which use liposomes designed to bind specific molecules (e.g., antibodies) and release encapsulated reagents or markers. The released chemicals are then detected according to standard techniques (see Monroe et al., Amer. Clin. Prod. Rev. 5:34-41 (1986)).

Reduction of Non-Specific Binding

One of skill in the art will appreciate that it is often desirable to minimize non-specific binding in immunoassays. Particularly, where the assay involves an antigen or antibody immobilized on a solid substrate it is desirable to minimize the amount of non-specific binding to the substrate. Means of reducing such non-specific binding are well known to those of skill in the art. Typically, this technique involves coating the substrate with a proteinaceous composition. In particular, protein compositions such as bovine serum albumin (BSA), nonfat powdered milk, and gelatin are widely used with powdered milk being most preferred.

Labels

The particular label or detectable group used in the assay is not a critical aspect of the invention, as long as it does not significantly interfere with the specific binding of the antibody used in the assay. The detectable group can be any material having a detectable physical or chemical property. Such detectable labels have been well-developed in the field of immunoassays and, in general, most any label useful in such methods can be applied to the present invention. Thus, a label is any composition detectable by spectroscopic, photochemical, biochemical, immunochemical, electrical, optical or chemical means. Useful labels in the present invention include magnetic beads (e.g., DYNABEADS™), fluorescent dyes (e.g., fluorescein isothiocyanate, Texas red, rhodamine, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold or colored glass or plastic beads (e.g., polystyrene, polypropylene, latex, etc.).

The label may be coupled directly or indirectly to the desired component of the assay according to methods well known in the art. As indicated above, a wide variety of labels may be used, with the choice of label depending on sensitivity required, ease of conjugation with the compound, stability requirements, available instrumentation, and disposal provisions.

Non-radioactive labels are often attached by indirect means. Generally, a ligand molecule (e.g., biotin) is covalently bound to the molecule. The ligand then binds to another molecule (e.g., streptavidin), which is either inherently detectable or covalently bound to a signal system, such as a detectable enzyme, a fluorescent compound, or a chemiluminescent compound. The ligands and their targets can be used in any suitable combination with antibodies that recognize GPCRs, or secondary antibodies that recognize anti-GPCR.

The molecules can also be conjugated directly to signal generating compounds, e.g., by conjugation with an enzyme or fluorophore. Enzymes of interest as labels will primarily be hydrolases, particularly phosphatases, esterases and glycosidases, or oxidotases, particularly peroxidases. Fluorescent compounds include fluorescein and its derivatives, rhodamine and its derivatives, dansyl, umbelliferone, etc. Chemiluminescent compounds include luciferin, and 2,3-dihydrophthalazinediones, e.g., luminol. For a review of various labeling or signal producing systems that may be used, see U.S. Pat. No. 4,391,904.

Means of detecting labels are well known to those of skill in the art. Thus, for example, where the label is a radioactive label, means for detection include a scintillation counter or photographic film as in autoradiography. Where the label is a fluorescent label, it may be detected by exciting the fluorochrome with the appropriate wavelength of light and detecting the resulting fluorescence. The fluorescence may be detected visually, by means of photographic film, by the use of electronic detectors such as charge coupled devices (CCDs) or photomultipliers and the like. Similarly, enzymatic labels may be detected by providing the appropriate substrates for the enzyme and detecting the resulting reaction product. Finally simple colorimetric labels may be detected simply by observing the color associated with the label. Thus, in various dipstick assays, conjugated gold often appears pink, while various conjugated beads appear the color of the bead.

Some assay formats do not require the use of labeled components. For instance, agglutination assays can be used to detect the presence of the target antibodies. In this case, antigen-coated particles are agglutinated by samples comprising the target antibodies. In this format, none of the components need be labeled and the presence of the target antibody is detected by simple visual inspection.

Assays for Modulators of GPCRs

A. Assays for GPCR Activity

GPCRs and their alleles and polymorphic variants are G-protein coupled receptors that participate in signal transduction and are associated with cellular function (e.g., detection of ligands) in a variety of cells, e.g., kidney, liver, colon, adipose, hypothalamus, and other cells. The activity of GPCR polypeptides can be assessed using a variety of in vitro and in vivo assays to determine functional, chemical, and physical effects, e.g., measuring ligand binding (e.g., radioactive ligand binding), second messengers (e.g., cAMP, cGMP, IP₃, DAG, or Ca²⁺), ion flux, phosphorylation levels, transcription levels, neurotransmitter levels, and the like. Furthermore, such assays can be used to test for inhibitors and activators of a GPCR. Modulators can also be genetically altered versions of a GPCR. Screening assays of the invention are used to identify modulators that can be used as therapeutic agents, e.g., antibodies to GPCRs and antagonists of GPCR activity.

The GPCR of the assay will be selected from a polypeptide having a sequence of SEQ ID NO:2; SEQ ID NO:4, SEQ ID NO:6, SEQ ID NO:8, SEQ ID NO:10, SEQ ID NO:12, SEQ ID NO:16, or SEQ ID NO:18 or conservatively modified variants thereof. Alternatively, the GPCR of the assay will be derived from a eukaryote and include an amino acid subsequence having amino acid sequence identity to SEQ ID NO:2; SEQ ID NO:4, SEQ ID NO:6, SEQ ID NO:8, SEQ ID NO:10, SEQ ID NO:12, SEQ i) NO:16, or SEQ ID NO:18. Generally, the amino acid sequence identity will be at least 70%, optionally at least 80%, optionally at least 90-95%. Optionally, the polypeptide of the assays will comprise a domain of a GPCR, such as an extracellular domain, transmembrane domain, cytoplasmic domain, ligand binding domain, subunit association domain, active site, and the like. Either a GPCR or a domain thereof can be covalently linked to a heterologous protein to create a chimeric protein used in the assays described herein.

Modulators of GPCR activity are tested using GPCR polypeptides as described above, either recombinant or naturally occurring. The protein can be isolated, expressed in a cell, expressed in a membrane derived from a cell, expressed in tissue or in an animal, either recombinant or naturally occurring. For example, kidney cells, liver cells, colon cells, transformed cells, or membranes can be used. Modulation is tested using one of the in vitro or in vivo assays described herein. Signal transduction can also be examined in vitro with soluble or solid state reactions, using a chimeric molecule such as an extracellular domain of a receptor covalently linked to a heterologous signal transduction domain, or a heterologous extracellular domain covalently linked to the transmembrane and or cytoplasmic domain of a receptor. Gene amplification can also be examined. Furthermore, ligand-binding domains of the protein of interest can be used in vitro in soluble or solid state reactions to assay for ligand binding.

Ligand binding to GPCR, a domain, or chimeric protein can be tested in solution, in a bilayer membrane, attached to a solid phase, in a lipid monolayer, or in vesicles. Binding of a modulator can be tested using, e.g., changes in spectroscopic characteristics (e.g., fluorescence, absorbance, refractive index) hydrodynamic (e.g., shape), chromatographic, or solubility properties.

Receptor-G-protein interactions can also be examined. For example, binding of the G-protein to the receptor or its release from the receptor can be examined. For example, in the absence of GTP, an activator will lead to the formation of a tight complex of a G protein (all three subunits) with the receptor. This complex can be detected in a variety of ways, as noted above. Such an assay can be modified to search for inhibitors. Add an activator to the receptor and G protein in the absence of GTP, form a tight complex, and then screen for inhibitors by looking at dissociation of the receptor-G protein complex. In the presence of GTP, release of the alpha subunit of the G protein from the other two G protein subunits serves as a criterion of activation.

An activated or inhibited G-protein will in turn alter the properties of downstream effectors such as proteins, enzymes, and channels. The classic examples are the activation of cGMP phosphodiesterase by transducin in the visual system, adenylate cyclase by the stimulatory G-protein, phospholipase C by Gq and other cognate G proteins, and modulation of diverse channels by Gi and other G proteins. Downstream consequences can also be examined such as generation of diacyl glycerol and IP3 by phospholipase C, and in turn, for calcium mobilization by IP₃.

Activated GPCR receptors become substrates for kinases that phosphorylate the C-terminal tail of the receptor (and possibly other sites as well). Thus, activators will promote the transfer of ³²P from gamma-labeled GTP to the receptor, which can be assayed with a scintillation counter. The phosphorylation of the C-terminal tail will promote the binding of arrestin-like proteins and will interfere with the binding of G-proteins. The kinase/arrestin pathway plays a key role in the desensitization of many GPCR receptors. For a general review of GPCR signal transduction and methods of assaying signal transduction, see, e.g., Methods in Enzymology, vols. 237 and 238 (1994) and volume 96 (1983); Bourne et al., Nature 10:349:117-27 (1991); Bourne et al., Nature 348:125-32 (1990); Pitcher et al., Annu. Rev. Biochem. 67:653-92 (1998).

Samples or assays that are treated with a potential GPCR inhibitor or activator are compared to control samples without the test compound, to examine the extent of modulation. Control samples (untreated with activators or inhibitors) are assigned a relative GPCR activity value of 100. Inhibition of a GPCR is achieved when the GPCR activity value relative to the control is about 90%, optionally 50%, optionally 25-0%. Activation of a GPCR is achieved when the GPCR activity value relative to the control is 110%, optionally 150%, 200-500%, or 1000-2000%.

Changes in ion flux may be assessed by determining changes in polarization (i.e., electrical potential) of the cell or membrane expressing a GPCR. One means to determine changes in cellular polarization is by measuring changes in current (thereby measuring changes in polarization) with voltage-clamp and patch-clamp techniques, e.g., the “cell-attached” mode, the “inside-out” mode, and the “whole cell” mode (see, e.g., Ackerman et al., New Engl. J. Med. 336:1575-1595 (1997)). Whole cell currents are conveniently determined using the standard methodology (see, e.g., Hamil et al., PFlugers. Archiv. 391:85 (1981). Other known assays include: radiolabeled ion flux assays and fluorescence assays using voltage-sensitive dyes (see, e.g., Vestergarrd-Bogind et al., J. Membrane Biol. 88:67-75 (1988); Gonzales & Tsien, Chem. Biol. 4:269-277 (1997); Daniel et al., J. Pharmacol. Meth..25:185-193 (1991); Holevinskyet al., J. Membrane Biology 137:59-70 (1994)). Generally, the compounds to be tested are present in the range from 1 pM to 100 mM.

The effects of the test compounds upon the function of the polypeptides can be measured by examining any of the parameters described above. Any suitable physiological change that affects GPCR activity can be used to assess the influence of a test compound on the polypeptides of this invention. When the functional consequences are determined using intact cells or animals, one can also measure a variety of effects such as transmitter release, hormone release, transcriptional changes to both known and uncharacterized genetic markers (e.g., northern blots), changes in cell metabolism such as cell growth or pH changes, and changes in intracellular second messengers such as Ca²⁺, IP3 or cAMP.

Preferred assays for G-protein coupled receptors include cells that are loaded with ion or voltage sensitive dyes to report receptor activity. Assays for determining activity of such receptors can also use known agonists and antagonists for other G-protein coupled receptors as negative or positive controls to assess activity of tested compounds. In assays for identifying modulatory compounds (e.g., agonists, antagonists), changes in the level of ions in the cytoplasm or membrane voltage will be monitored using an ion sensitive or membrane voltage fluorescent indicator, respectively. Among the ion-sensitive indicators and voltage probes that may be employed are those disclosed in the Molecular Probes 1997 Catalog. For G-protein coupled receptors, promiscuous G-proteins such as Gα15 and Gα16 can be used in the assay of choice (Wilkie et al., Proc. Nat'l Acad. Sci. USA 88:10049-10053 (1991)). Such promiscuous G-proteins allow coupling of a wide range of receptors to signal transduction pathways in heterologous cells.

Receptor activation typically initiates subsequent intracellular events, e.g., increases in second messengers such as IP3, which releases intracellular stores of calcium ions. Activation of some G-protein coupled receptors stimulates the formation of inositol triphosphate (IP₃) through phospholipase C-mediated hydrolysis of phosphatidylinositol (Berridge & Irvine, Nature 312:315-21 (1984)). IP3 in turn stimulates the release of intracellular calcium ion stores. Thus, a change in cytoplasmic calcium ion levels, or a change in second messenger levels such as IP3 can be used to assess G-protein coupled receptor function. Cells expressing such G-protein coupled receptors may exhibit increased cytoplasmic calcium levels as a result of contribution from both intracellular stores and via activation of ion channels, in which case it may be desirable although not necessary to conduct such assays in calcium-free buffer, optionally supplemented with a chelating agent such as EGTA, to distinguish fluorescence response resulting from calcium release from internal stores.

Other assays can involve determining the activity of receptors which, when activated, result in a change in the level of intracellular cyclic nucleotides, e.g., cAMP or cGMP, by activating or inhibiting downstream effectors such as adenylate cyclase. There are cyclic nucleotide-gated ion channels, e.g., rod photoreceptor cell channels and olfactory neuron channels that are permeable to cations upon activation by binding of cAMP or cGMP (see, e.g., Altenhofen et al., Proc. Natl. Acad. Sci. U.S.A. 88:9868-9872 (1991) and Dhallan et al., Nature 347:184-187 (1990)). In cases where activation of the receptor results in a decrease in cyclic nucleotide levels, it may be preferable to expose the cells to agents that increase intracellular cyclic nucleotide levels, e.g., forskolin, prior to adding a receptor-activating compound to the cells in the assay. Cells for this type of assay can be made by co-transfection of a host cell with DNA encoding a cyclic nucleotide-gated ion channel, GPCR phosphatase and DNA encoding a receptor (e.g., certain glutamate receptors, muscarinic acetylcholine receptors, dopamine receptors, serotonin receptors, and the like), which, when activated, causes a change in cyclic nucleotide levels in the cytoplasm.

In one embodiment, changes in intracellular cAMP or cGMP can be measured using immunoassays. The method described in Offermanns & Simon, J. Biol. Chem. 270:15175-15180 (1995) may be used to determine the level of cAMP. Also, the method described in Felley-Bosco et al., Am. J. Resp. Cell and Mol. Biol. 11:159-164 (1994) may be used to determine the level of cGMP. Further, an assay kit for measuring cAMP and/or cGMP is described in U.S. Pat. No. 4,115,538, herein incorporated by reference.

In another embodiment, phosphatidyl inositol (PI) hydrolysis can be analyzed according to U.S. Pat. No. 5,436,128, herein incorporated by reference. Briefly, the assay involves labeling of cells with ³H-myoinositol for 48-or more hrs. The labeled cells are treated with a test compound for one hour. The treated cells are lysed and extracted in chloroform-methanol-water after which the inositol phosphates were separated by ion exchange chromatography and quantified by scintillation counting. Fold stimulation is determined by calculating the ratio of cpm in the presence of agonist to cpm in the presence of buffer control. Likewise, fold inhibition is determined by calculating the ratio of cpm in the presence of antagonist to cpm in the presence of buffer control (which may or may not contain an agonist).

In another embodiment, transcription levels can be measured to assess the effects of a test compound on signal transduction. A host cell containing the protein of interest is contacted with a test compound for a sufficient time to effect any interactions, and then the level of gene expression is measured. The amount of time to effect such interactions may be empirically determined, such as by running a time course and measuring the level of transcription as a function of time. The amount of transcription may be measured by using any method known to those of skill in the art to be suitable. For example, mRNA expression of the protein of interest may be detected using northern blots or their polypeptide products may be identified using immunoassays. Alternatively, transcription based assays using reporter gene may be used as described in U.S. Pat. No. 5,436,128, herein incorporated by reference. The reporter genes can be, e.g., chloramphenicol acetyltransferase, firefly luciferase, bacterial luciferase, β-galactosidase and alkaline phosphatase. Furthermore, the protein of interest can be used as an indirect reporter via attachment to a second reporter such as green fluorescent protein (see, e.g.. Mistili & Spector, Nature Biotechnology 15:961-964 (1997)).

The amount of transcription is then compared to the amount of transcription in either the same cell in the absence of the test compound, or it may be compared with the amount of transcription in a substantially identical cell that lacks the protein of interest. A-substantially identical cell may be derived from the same cells from which the recombinant cell was prepared but which had not been modified by introduction of heterologous DNA. Any difference in the amount of transcription indicates that the test compound has in some manner altered the activity of the protein of interest.

B. Modulators

The compounds tested as modulators of GPCRs can be any small chemical compound, or a biological entity, e.g., a macromolecule such as a protein, sugar, nucleic acid or lipid. Alternatively, modulators can be genetically altered versions of a GPCR. Typically, test compounds will be small chemical molecules and peptides. Essentially any chemical compound can be used as a potential modulator or ligand in the assays of the invention, although most often compounds can be dissolved in aqueous or organic (especially DMSO-based) solutions. The assays are designed to screen large chemical libraries by automating the assay steps-and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland) and the like.

In one preferred embodiment, high throughput screening methods involve providing a combinatorial chemical or peptide library containing a large number of potential therapeutic compounds (potential modulator or ligand compounds). Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Sambrook, all supra), peptide nucleic acid libraries (see, e.g. U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, Jan 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Tripos, Inc., St. Louis, Mo., 3D Pharmaceuticals, Exton, Pa., Martek Biosciences, Columbia, Md., etc.).

C. Solid State and Soluble High Throughput Assays

In one embodiment the invention provides soluble assays using molecules such as a domain such as ligand binding domain, an extracellular domain, a transmembrane domain (e.g., one comprising seven transmembrane regions and cytosolic loops), the transmembrane domain and a cytoplasmic domain, an active site, a subunit association region, etc.; a domain that is covalently linked to a heterologous protein to create a chimeric molecule; a GPCR; or a cell or tissue expressing a GPCR, either naturally occurring or recombinant. In another embodiment, the invention provides solid phase based in vitro assays in a high throughput format, where the domain, chimeric molecule, GPCR, or cell or tissue expressing a GPCR is attached to a solid phase substrate.

In the high throughput assays of the invention, it is possible to screen up to several thousand different modulators or ligands in a single day. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (e.g., 96) modulators. If 1536 well plates are used, then a single plate can easily assay from about 100- about 1500 different compounds. It is possible to assay several different plates per day; assay screens for up to about 6,000-20,000 different compounds is possible using the integrated systems of the invention.

The molecule of interest can be bound to the solid state component, directly or indirectly, via covalent or non covalent linkage e.g., via a tag. The tag can be any of a variety of components. In general, a molecule which binds the tag (a tag binder) is fixed to a solid support, and the tagged molecule of interest (e.g., the signal transduction molecule of interest) is attached to the solid support by interaction of the tag and the tag binder.

A number of tags and tag binders can be used, based upon known molecular interactions well described in the literature. For example, where a tag has a natural binder, for example, biotin, protein A, or protein G, it can be used in conjunction with appropriate tag binders (avidin, streptavidin, neutravidin, the Fc region of an immunoglobulin, etc.) Antibodies to molecules with natural binders such as biotin are also widely available and appropriate tag binders; see, SIGMA Immunochemicals 1998 catalogue SIGMA, St. Louis Mo.).

Similarly, any haptenic or antigenic compound can be used in combination with an appropriate antibody to form a tag/tag binder pair. Thousands of specific antibodies are commercially available and many additional antibodies are described in the literature. For example, in one common configuration, the tag is a first antibody and the tag binder is a second antibody which recognizes the first antibody. In addition to antibody-antigen interactions, receptor-ligand interactions are also appropriate as tag and tag-binder pairs. For example, agonists and antagonists of cell membrane receptors (e.g., cell receptor-ligand interactions such as transferrin, c-kit, viral receptor ligands, cytokine receptors, chemokine receptors, interleukin receptors, immunoglobulin receptors and antibodies, the cadherein family, the integrin family, the selectin family, and the like; see, e.g., Pigott & Power, The Adhesion Molecule Facts Book I (1993). Similarly, toxins and venoms, viral epitopes, hormones (e.g., opiates, steroids, etc.), intracellular receptors (e.g. which mediate the effects of various small ligands, including steroids, thyroid hormone, retinoids and vitamin D; peptides), drugs, lectins, sugars, nucleic acids (both linear and cyclic polymer configurations), oligosaccharides, proteins, phospholipids and antibodies can all interact with various cell receptors.

Synthetic polymers, such as polyurethanes, polyesters, polycarbonates, polyureas, polyamides, polyethyleneimines, polyarylene sulfides, polysiloxanes, polyimides, and polyacetates can also form an appropriate tag or tag binder. Many other tag/tag binder pairs are also useful in assay systems described herein, as would be apparent to one of skill upon review of this disclosure.

Common linkers such as peptides, polyethers, and the like can also serve as tags, and include polypeptide sequences, such as poly-gly sequences of between about 5 and 200 amino acids. Such flexible linkers are known to persons of skill in the art. For example, poly(ethelyne glycol) linkers are available from Shearwater Polymers, Inc. Huntsville, Ala. These linkers optionally have amide linkages, sulfhydryl linkages, or heterofunctional linkages.

Tag binders are fixed to solid substrates using any of a variety of methods currently available. Solid substrates are commonly derivatized or functionalized by exposing all or a portion of the substrate to a chemical reagent which fixes a chemical group to the surface which is reactive with a portion of the tag binder. For example, groups which are suitable for attachment to a longer chain portion would include amines, hydroxyl, thiol, and carboxyl groups. Aminoalkylsilanes and hydroxyalkylsilanes can be used to functionalize a variety of surfaces, such as glass surfaces. The construction of such solid phase biopolymer arrays is well described in the literature. See, e.g., Merrifield, J. Am. Chem. Soc. 85:2149-2154 (1963) (describing solid phase synthesis of, e.g., peptides); Geysen et al., J. Immun. Meth. 102:259-274 (1987) (describing synthesis of solid phase components on pins); Frank & Doring, Tetrahedron 44:60316040 (1988) (describing synthesis of various peptide sequences on cellulose disks); Fodor et al., Science, 251:767-777 (1991); Sheldon et al., Clinical Chemistry 39(4):718-719 (1993); and Kozal et al., Nature Medicine 2(7):753759 (1996) (all describing arrays of biopolymers fixed to solid substrates). Non-chemical approaches for fixing tag binders to substrates include other common methods, such as heat, cross-linking by UV radiation, and the like.

D. Computer-Based Assays

Yet another assay for compounds that modulate GPCR activity involves computer assisted drug design, in which a computer system is used to generate a three-dimensional structure of GPCR based on the structural information encoded by the amino acid sequence. The input amino acid sequence interacts directly and actively with a preestablished algorithm in a computer program to yield secondary, tertiary, and quaternary structural models of the protein. The models of the protein structure are then examined to identify regions of the structure that have the ability to bind, e.g., ligands. These regions are then used to identify ligands that bind to the protein.

The three-dimensional structural model of the protein is generated by entering protein amino acid sequences of at least 10 amino acid residues or corresponding nucleic acid sequences encoding a GPCR polypeptide into the computer system. The amino acid sequence of the polypeptide or the nucleic acid encoding the polypeptide is selected from the group consisting of SEQ ID NO:1; SEQ ID NO:3, SEQ ID NO:5, SEQ ID NO:7, SEQ ID NO:9, SEQ ID NO:11, SEQ ID NO:15, or SEQ ID NO:17; and SEQ ID NO:2; SEQ ID NO:4, SEQ ID NO:6, SEQ ID NO:8, SEQ ID NO:10, SEQ ID NO:12, SEQ ID NO:16, or SEQ ID NO:18, respectively, and conservatively modified versions thereof. The amino acid sequence represents the primary sequence or subsequence of the protein, which encodes the structural information of the protein. At least 10 residues of the amino acid sequence (or a nucleotide sequence encoding 10 amino acids) are entered into the computer system from computer keyboards, computer readable substrates that include, but are not limited to, electronic storage media (e.g., magnetic diskettes, tapes, cartridges, and chips), optical media (e.g., CD ROM), information distributed by internet sites, and by RAM. The three-dimensional structural model of the protein is then generated by the interaction of the amino acid sequence and the computer system, using software known to those of skill in the art.

The amino acid sequence represents a primary structure that encodes the information necessary to form the secondary, tertiary and quaternary structure of the protein of interest. The software looks at certain parameters encoded by the primary sequence to generate the structural model. These parameters are referred to as “energy terms,” and primarily include electrostatic potentials, hydrophobic potentials, solvent accessible surfaces, and hydrogen bonding. Secondary energy terms include van der Waals potentials. Biological molecules form the structures that minimize the energy terms in a cumulative fashion. The computer program is therefore using these terms encoded by the primary structure or amino acid sequence to create the secondary structural model.

The tertiary structure of the protein encoded by the secondary structure is then formed on the basis of the energy terms of the secondary structure. The user at this point can enter additional variables such as whether the protein is membrane bound or soluble, its location in the body, and its cellular location, e.g., cytoplasmic, surface, or nuclear. These variables along with the energy terms of the secondary structure are used to form the model of the tertiary structure. In modeling the tertiary structure, the computer program matches hydrophobic faces of secondary structure with like, and hydrophilic faces of secondary structure with like.

Once the structure has been generated, potential ligand binding regions are identified by the computer system. Three-dimensional structures for potential ligands are generated by entering amino acid or nucleotide sequences or chemical formulas of compounds, as described above. The three-dimensional structure of the potential ligand is then compared to that of the GPCR protein to identify ligands that bind to GPCR. Binding affinity between the protein and ligands is determined using energy terms to determine which ligands have an enhanced probability of binding to the protein.

Computer systems are also used to screen for mutations, polymorphic variants, alleles and interspecies homologs of GPCR genes. Such mutations can be associated with disease states or genetic traits. As described above, GeneChip™ and related technology can also be used to screen for mutations, polymorphic variants, alleles and interspecies homologs. Once the variants are identified, diagnostic assays can be used to identify patients having such mutated genes. Identification of the mutated GPCR genes involves receiving input of a first nucleic acid or amino acid sequence encoding an GPCR, selected from the group consisting of SEQ ID NO:1; SEQ ID NO:3, SEQ ID NO:5, SEQ ID NO:7, SEQ ID NO:9, SEQ ID NO:11, SEQ ID NO:15, or SEQ ID NO:17; and SEQ ID NO:2; SEQ ID NO:4, SEQ ID NO:6, SEQ ID NO:8, SEQ ID NO:10, SEQ ID NO:12, SEQ ID NO:16, or SEQ ID NO:18, respectively, and conservatively modified versions thereof. The sequence is entered into the computer system as described above. The first nucleic acid or amino acid sequence is then compared to a second nucleic acid or amino acid sequence that has substantial identity to the first sequence. The second sequence is entered into the computer system in the manner described above. Once the first and second sequences are compared, nucleotide or amino acid differences between the sequences are identified. Such sequences can represent allelic differences in GPCR genes, and mutations associated with disease states and genetic traits.

Transgenic Animals

Transgenic animals, including knockout transgenic animals, that include additional copies of the GPCRs of the invention and/or altered or mutated GPCR transgenes can also be generated. These animals can be used, for example, to screen for in vivo modulators of the GPCRs disclosed herein to develop therapies for TGR-related disorders. A “transgenic animal” refers to any animal (e.g. mouse, rat, pig, bird, or an amphibian), preferably a non-human mammal in which one or more cells contain heterologous nucleic acid introduced using transgenic techniques well known in the art. The nucleic acid is introduced into the cell, directly or indirectly, by introduction into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. This molecule may be integrated within a chromosome, or it may be extrachromosomally replicating DNA. In some embodiments, GPCR transgenic animals express a GPCR gene, which may have an altered function with respect to a normal GPCR gene.

In other embodiments, transgenic animals are produced in which the GPCR expression is silenced. Gene knockout by homologous recombination is a method that is commonly used to generate transgenic animals. This method requires a relatively long genomic clone of the gene to be knocked out (ca. 10 kb). Typically, a selectable marker is inserted into an exon of the gene of interest to effect the gene disruption, and a second counter-selectable marker provided outside of the region of homology to select homologous versus non-homologous recombinants. This construct is transfected into embryonic stem cells and recombinants selected in culture. Recombinant stem cells are combined with very early stage embryos generating chimeric animals. If the chimerism extends to the germline homozygous knockout animals can be isolated by back-crossing.

Other methods of generating knockout animals are typified by an approach using the cre recombinase and lox DNA recognition elements. The recognition elements are inserted into a gene of interest using homologous recombination (as described above) and the expression of the recombinase induced in adult mice post-development. This causes the deletion of a portion of the target gene and typically avoids developmental complications. Transgenic mice can be derived using methodology known to those of skill in the art, see, e.g., Hogan et al., Manipulating the Mouse Embryo: A Laboratory Manual, (1988); Teratocarcinomas and Embryonic Stem Cells: A Practical Approach, Robertson, ed., (1987); and Capecchi et al., Science 244:1288 (1989).

Kits

GPCRs and their homologs are a useful tool for identifying cells such as brain, e.g., hypothalamus, pancreas, retina, kidney, liver, adipose, or spleen cells, for forensics and paternity determinations, for diagnosing diseases, and for examining signal transduction. GPCR specific reagents that specifically hybridize to GPCR nucleic acids, such as GPCR probes and primers, and GPCR specific reagents that specifically bind to a GPCR protein, e.g., GPCR antibodies are used to examine signal transduction regulation.

Nucleic acid assays for the presence of GPCR DNA and RNA in a sample include numerous techniques are known to those skilled in the art, such as Southern analysis, northern analysis, dot blots, RNase protection, S1 analysis, amplification techniques such as PCR and LCR, and in situ hybridization. In in situ hybridization, for example, the target nucleic acid is liberated from its cellular surroundings in such a way as to be available for hybridization within the cell while preserving the cellular morphology for subsequent interpretation and analysis. The following articles provide an overview of the art of in situ hybridization: Singer et al., Biotechniques 4:230-250 (1986); Haase et al., Methods in Virology, vol. VII, pp. 189-226 (1984); and Nucleic Acid Hybridization: A Practical Approach (Hames et al., eds. 1987). In addition, GPCR protein can be detected with the various immunoassay techniques described above. The test sample is typically compared to both a positive control (e.g., a sample expressing a recombinant GPCR) and a negative control.

The present invention also provides for kits for screening for modulators of GPCRs. Such kits can be prepared from readily available materials and reagents. For example, such kits can comprise any one or more of the following materials: a GPCR, reaction tubes, and instructions for testing GPCR activity. Optionally, the kit contains biologically active GPCR. A wide variety of kits and components can be prepared according to the present invention, depending upon the intended user of the kit and the particular needs of the user.

Disease Treatment and Diagnosis

TGRs are involved in the regulation of many important physiological functions and are often therapeutic targets for various diseases or conditions. Mammalian TGRs are typically classified in three categories, class A, receptors related to rhodopsin and the adrenergic receptors, class B, receptors related to the calcitonin and parathyroid hormone receptors, and class C, receptors related to the metabotropic receptors. The rhodopsin/adrenergic receptor class is the largest class and includes various amine receptor, e.g., acetylcholine (muscarinic) receptors, adrenergic receptors, dopamine receptors, histamine receptors, serotonin receptors, and octopamine receptors; peptide receptors, e.g., angiotensin, bombesin, bradykinin, endothelin, interleukin-8, chemokine, melanocortin, neuropeptide Y, neurotensin, opioid, somatostatin, tachykinin, thrombin, vasopressin, galanin, proteinase-activated, orexin, and chemokine/chemotatic factor receptors; protein hormone receptors, e.g., FSH, lutropin-choriogonadotropic hormone, and thyrotropin receptors; rhodopsin receptors; olfactory receptors; prostanoid receptors; nucleotide-like receptors, including adenosine and purinoceptors; cannabis receptors; platelet activating factor receptor; gonadotropin-releasing hormone receptor; melatonin receptor, lysosphingolipid and LPA (EDG) receptors, as well as various orphan receptors. Class B includes calcitonin, corticotropin releasing factor, gastric inhibitory peptide glucagon, growth hormone-releasing hormone, parathyroid hormone, PACAP, secretin, vasoactive intestinal polypeptide, and brain-specific angiogenesis inhibitor receptors, among others. Class C receptors include metabotropic glutamate receptors and GABA-B subtype receptors as well as putative pheromone receptors.

Class A GPCRs function in a variety of physiological processes such as vasodilation, bronchodilation, neurotransmitter signaling, stimulation of endocrine secretions, gut peristalsis, development, mitogenesis, cell proliferation, cell migration, immune system function, and oncogenesis. Accordingly, class A GPCRs can be used, for example, as probes to identify cells or tissues that exhibit dysregulation of these processes, and moreover, as screening targets to identify modulators of these processes.

Class B GPCRs also function in a wide range of physiological processes such as regulation of calcium homeostasis, modulation of activity of cells in the immune system, various excitatory and inhibitory actions in the central nervous system, control of smooth muscle relaxation, control of smooth muscle, secretion in stomach, intestinal epithelium, pancreas, and gall bladder. Accordingly, class B GPCRs can be used, for example, as probes to identify cells or tissues that exhibit dyregulation of these process, and to identify modulators of these physiological processes.

Class C GPCRs, metabotropic glutamate receptors, are also important regulators of physiological processes such as neurotransmission. Glutamate is the major neurotransmitter in the CNS and plays an important role in neuronal plasticity, cognition, memory, learning, and some neurological disorders such as epilepsy, stroke, and neurodegeneration. B-type receptors for the neurotransmitter GABA (gamma-aminobutyric acid) inhibit neuronal activity through G-protein-coupled second-messenger systems, which regulate the release of neurotransmitters and the activity of ion channels and adenylyl cyclase. Thus, GABA B-type receptors play a role in controlling neuronal function and are also involved in such processes as neuronal plasticity, cognition, memory, and learning. Accordingly, class C GPCRs can be used, for example, as probes to identify cells or tissues, particularly, neuronal cells or tissues, that exhibit dysregulation of these processes, and to identify modulators of these physiological processes for the treatment of neurological disorders.

In certain embodiments, the presently-described GPCRs can be used in the diagnosis and treatment of certain diseases or conditions, i.e., TGR-associated disorders. For example, the activity of GPCRs (e.g., TGR-342, -60, -346, or -339) that are expressed or preferentially expressed in a particular cell type, can be used to modulate cellular function (e.g., responsiveness to extracellular signals), thereby specifically modulating the function of the cells of that type in a patient. Further, mutations in the cell specific GPCRs will likely produce a disease, condition, or symptom associated with a lack of function of the particular cell type. For example, mutations in GPCRs preferentially expressed in the hypothalamus, e.g., TGR60, TGR342, TGR346, will likely result in any number of conditions associated with the hypothalamus and the pituitary gland, which is often controlled by chemical mediators secreted by the hypothalamus. Dysfunction of hypothalamus-specific GPCRs can, for example, alter secretion of one or more hypothalamic factors such as growth hormone-releasing hormone, somatostatin, gonadotropin-releasing hormone, thyrotropin-releasing hormone, and corticotropin-releasing hormone. Thus, hypothalamic-associated diseases include hypothyroidism, hypogonadism, growth disorders, and hyperprolactinemia, as well as diabetes insipidus, and disturbances of thirst, sleep, temperature regulation, appetite, blood pressure or any other syndrome or disease associated with the hypothalamus (see, e.g., Harrison's Principles of Internal Medicine, 12th Edition, Wilson, et al., eds., McGraw-Hill, Inc.), e.g., endocrine processes and autonomic nervous system function.

In certain embodiments, the presently-described GPCRs can be used in the diagnosis and treatment of diseases or conditions. For example, the activity of GPCRs that are expressed in a particular cell type can be used to modulate cellular function (e.g., responsiveness to extracellular signals), thereby specifically modulating the function of the cells of that type in a patient. Further, mutations in the cell specific GPCRs will likely produce a disease, condition, or symptom associated with a lack of function of the particular cell type. For example, kidney-specific GPCRs will likely result in any of a number of nephrotic conditions or diseases, such as renal failure, nephritis, nephrotic syndrome, asymptomatic urinary abnormalities, renal tubule defects, hypertension, nephrolithiasis, or any other syndrome or disease associated with the kidneys (see, e.g., Harrison's Principles of Internal Medicine, 12th Edition, Wilson, et al., eds., McGraw-Hill, Inc.).

Similarly, mutations in liver-specific GPCRs can be used to diagnose any liver-related disease or condition,; e.g., cirrhosis, infiltrations, lesions, functional disorders, and jaundice. Mutations in adipocyte-specific genes can also be used to detect, or diagnose a propensity for, conditions such as obesity. Other conditions associated with any of the herein-provided GPCRs include, e.g., hyperlipidemia or endocrine disorders. Mutations in spleen-specific GPCRs can result in any spleen-associated disorder or condition, e.g., splenic enlargement, immune disorders, blood disorders, and others. Mutations or abnormal function, or level, of pancreas-associated GPCRs may play a role in diseases associated with failure to control blood glucose levels such as diabetes. GPCRs expressed in the lung may be involved in disorders of pulmonary function. Mutations or dysregulation of TGRs expressed in lymphocytes or hematopoietic cell-associated TGRs, i.e., TGRs preferentially expressed in peripheral blood lymphocytes (PBLs), bone marrow, thymus, or hematopoietic cell lineages including cells involved in the immune system, can lead to malignancies, anemia, and other disorders of immune function such as autoimmune diseases. Mutations in colon-specific GPCRs can result in any colon-associated condition or disease, e.g., alterations in bowel habit, rectal bleeding, pain, and other symptoms. The diseases listed herein as well as many other diseases of various organs, tissues, or cells that can be associated with GPCR dysfunction are described, e.g., in Harrison's, supra).

Accordingly, the present sequences can be used to diagnose any of the herein-described disorders or conditions in a patient, e.g., by examining the sequence, level, or activity of any of the present GPCRs in a patient, wherein an alteration, e.g., a decrease, in the level of expression or activity in a GPCR, or the detection of a deleterious mutation in a GPCR, indicates the presence or the likelihood of the disease or condition. Similarly, modulation of the present GPCRs (e.g., by administering modulators of the GPCR) can be used to treat or prevent any of the conditions or diseases.

TGR342's Role as a Melanin-Concentrating Hormone Ligand

The present invention is particularly important in that it demonstrates, for the first time, that a second receptor, TGR342 or, alternatively, MCHR2, exists for the ligand MCH. It is thought that both receptors are important for the MCH-related phenotypes that have been observed. Likewise, it is thought that both receptors could have unique and/or common functions associated with MCH. Compounds that affect one or both receptors can now be developed and screened. In addition to the use of such compounds for studying the function of these two receptors and MCH functions, the compounds will also be useful in the diagnosis, treatment and prevention of diseases and conditions associated with abnormal MCH function.

The entire general description regarding GPCRs, GPCR function, and modulation thereof is applicable to TGR342 and its role as a MCH receptor (i.e., MCHR2). Thus, as alluded to above, the invention provides a method of identifying a modulator of MCHR2. One aspect of the present invention entails a method for identifying a modulator of MCHR2 activity comprising contacting an MCHR2 with a candidate modulator and determining whether the candidate modulator produces a functional effect on the MCHR2. The modulator of MCHR2 can be any type of molecule; in preferred embodiments, it is a compound or an antibody. In certain embodiments, the modulator is an agonist of MCHR2 activity, while it is an antagonist of MCHR2 activity in other preferred embodiments. The modulator may exert its effect directly on MCHR2, or indirectly through, for example, a binding partner. The functional effect may be any type of effect previously described, including a chemical effect or a physical effect. In preferred embodiments, the functional effect is determined by measuring changes in intracellular cAMP, IP3 or calcium.

The present invention also encompasses methods of modulating MCHR2-associated activity in a mammal, the method comprising administering to the mammal an agonist or antagonist of MCHR-2 associated activity in an amount sufficient to modulate such activity. More specifically, the present invention contemplates methods of treating, preventing or diagnosing diseases in a mammal with an agonist or antagonist of MCHR2-associated activity, the methods comprising administering the agonist or antagonist to a mammal in an amount sufficient to agonize or antagonize MCHR2 associated functions. In particular diagnostic embodiments, the diagnostic methods of the invention contemplate identification of a mammal having or susceptible to an MCHR2-associated condition, by detecting MCHR2 nucleic acids or polypeptides in a sample.

The invention also provides a method of identifying an MCHR2 ligand, the method comprising contacting an MCHR2 with a candidate ligand under conditions that allow for selective binding between the MCHR2 and a MCHR2 ligand, and identifying a ligand that selectively binds to the MCHR2.

As previously discussed, a tremendous body of evidence exists that demonstrates that MCH plays an integral role as a neurotransmitter/neuroregulator in a variety of behavioral responses. Potentially, the most prominent role for MCH is its regulatory role in feeding behavior and energy balance (see, Flier and Maratos-Flier, Cell 92:437-40 (1998) and Qu et al., Nature 380:243-47 (1996)).

It is known that MCHR, the first-identified receptor for MCH (see, International Application No. PCT/US00/15503, hereby incorporated by reference) is expressed in brain, at moderate levels in the eye and skeletal muscle, and in low levels in tongue and the pituitary gland. These findings indicate that MCH is likely to play a role in, inter alia, olfactory learning, regulation of feeding behavior and energy metabolism, regulation of the hypothalmic-pituitary-adrenocortical axis following stress, arousal and the sensation of anxiety (see, Saito et al., TEM 11(8):299-303 (2000)).

As noted above and discussed in Example 2, TGR342 (MCHR2) has been detected in the following locations (the number of “+” represents the intensity of the signal): paracentral gyrus of cerebral cortex (involved in, for example, movement control; ++++); the parietal lobe of cerebral cortex (involved in, for example, somatic sensation; ++++); the temporal lobe of cerebral cortex (involved in, for example, hearing, learning, memory and emotion; +++), the frontal lobe of cerebral cortex (involved in, for example, movement control; ++); the occipital lobe of cerebral cortex (involved in, for example, vision; ++); accumbens nucleus (involved in, for example, feeling, receiving and integrating input from other elements involved in neurological function, including amygdala, hippocampus, and the temporal lobe; +++); hippocampus (involved in, for example, memory and emotion; ++); putamen (involved in, for example, movement control; ++); amygdala (involved in, for example, emotion (learning, fear and pleasure); +); caudate nucleus (involved in, for example, movement control; +); and pons (involved in, for example, relaying information regarding movement and sensation from the cerebral cortex to the cerebellum; +). TGR342 (MCHR2) has also been detected in whole brain (++) and fetal brain (++) and is believed to be present in the hypothalamus (involved in, for example, integrating autonomic response with behavior).

The information known regarding MCHR-associated conditions (see, e.g., Chambers et al., Nature 400:261-65 (1999); Saito et al., Nature 400:265-69 (1999); Saito et al., TEM 11(8):299-303 (2000); and International Application No. PCT/US00/15503) coupled with the information set forth herein strongly suggests that TGR342 (MCHR2) is involved in a diverse range of functions. The present invention specifically contemplates the modulation of TGR342 function, and thus the modulation of conditions associated with those functions. Such conditions include, but are not limited to, disorders relating to feeding behavior and weight control, movement, mood and behavior (e.g., anxiety), and learning and memory (e.g., association). It is thought that disorders relating to sleep, growth dopaminergic system function and reproductive function are also affected by modulation of TGR342 function. Such modulation can be used in the diagnosis, prevention, and treatment of disorders relating to MCHR2 in mammals, particularly humans.

TGR342 is mapped to human chromosome 6q21 to the same locus as the Non-MHC susceptibility locus to type 1 diabetes (IDDM15). TGR342 is expressed in the pancreas and its ligand, MCH, has been shown to stimulate insulin secretion. Thus, TGR342 nucleic acids and polypeptides may be used for the diagnosis and/or prognosis of diabetes. Furthermore, modulators of TGR342 may be used for the treatment of diabetes.

Administration and Pharmaceutical Compositions

GPCR modulators can be administered directly to the mammalian subject for modulation of signal transduction in vivo, e.g., for the treatment of any of the diseases or conditions described supra. Administration is by any of the routes normally used for introducing a modulator compound into ultimate contact with the tissue to be treated. The GPCR modulators are administered in any suitable manner, optionally with pharmaceutically acceptable carriers. Suitable methods of administering such modulators are available and well known to those of skill in the art, and, although more than one route can be used to administer a particular composition, a particular route can often provide a more immediate and more effective reaction than another route.

Pharmaceutically acceptable carriers are determined in part by the particular composition being administered, as well as by the particular method used to administer the composition. Accordingly, there is a wide variety of suitable formulations of pharmaceutical compositions of the present invention (see, e.g., Remington 's Pharmaceutical Sciences, 17th ed. 1985)).

The GPCR modulators, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Formulations suitable for administration include aqueous and non-aqueous solutions, isotonic sterile solutions, which can contain antioxidants, buffers, bacteriostats, and solutes that render the formulation isotonic, and aqueous and non-aqueous sterile suspensions that can include suspending agents, solubilizers, thickening agents, stabilizers, and preservatives. In the practice of this invention, compositions can be administered, for example, by orally, topically, intravenously, intraperitoneally, intravesically or intrathecally. Optionally, the compositions are administered orally or nasally. The formulations of compounds can be presented in unit-dose or multi-dose sealed containers, such as ampules and vials. Solutions and suspensions can be prepared from sterile powders, granules, and tablets of the kind previously described. The modulators can also be administered as part a of prepared food or drug.

The dose administered to a patient, in the context of the present invention should be sufficient to effect a beneficial response in the subject over time. Such doses are administered prophylactically or to an individual already suffering from the disease. The compositions are administered to a patient in an amount sufficient to elicit an effective protective or therapeutic response in the patient. An amount adequate to accomplish this is defined as “therapeutically effective dose.” The dose will be determined by the efficacy of the particular GPCR modulators (e.g., GPCR antagonists and anti-GPCR antibodies) employed and the condition of the subject, as well as the body weight or surface area of the area to be treated. The size of the dose also will be determined by the existence, nature, and extent of any adverse side-effects that accompany the administration of a particular compound or vector in a particular subject.

In determining the effective amount of the modulator to be administered, a physician may evaluate circulating plasma levels of the modulator, modulator toxicities, and the production of anti-modulator antibodies. In general, the dose equivalent of a modulator is from about 1 ng/kg to 10 mg/kg for a typical subject.

For administration, GPCR modulators of the present invention can be administered at a rate determined by the LD-50 of the modulator, and the side-effects of the inhibitor at various concentrations, as applied to the mass and overall health of the subject. Administration can be accomplished via single or divided doses.

### EXAMPLES

The following examples are provided by way of illustration only and not by way of limitation. Those of skill in the art will readily recognize a variety of noncritical parameters that could be changed or modified to yield essentially similar results.

### Example 1

### Isolation of Full-Length TGR342 cDNA

A novel GPCR was identified by searching the public databases (Swiss-Prot and Genbank). Its nucleic acid sequence is provided in SEQ ID NO:1, while its deduced amino acid sequence is provided in SEQ ID NO:2. The novel GPCR, designated TGR342, is available under the accession number AC027643 (Genbank).

A GPCR profile homology search of the human genomic databases set forth above (Swiss-Prot and Genbank) revealed a partial reading frame for a peptide that has significant homology to MCH and somatostatin receptor proteins. The full-length TGR342 cDNA was cloned by 5′ and 3′ racing PCR using a Clontech Marathon Racing Kit.

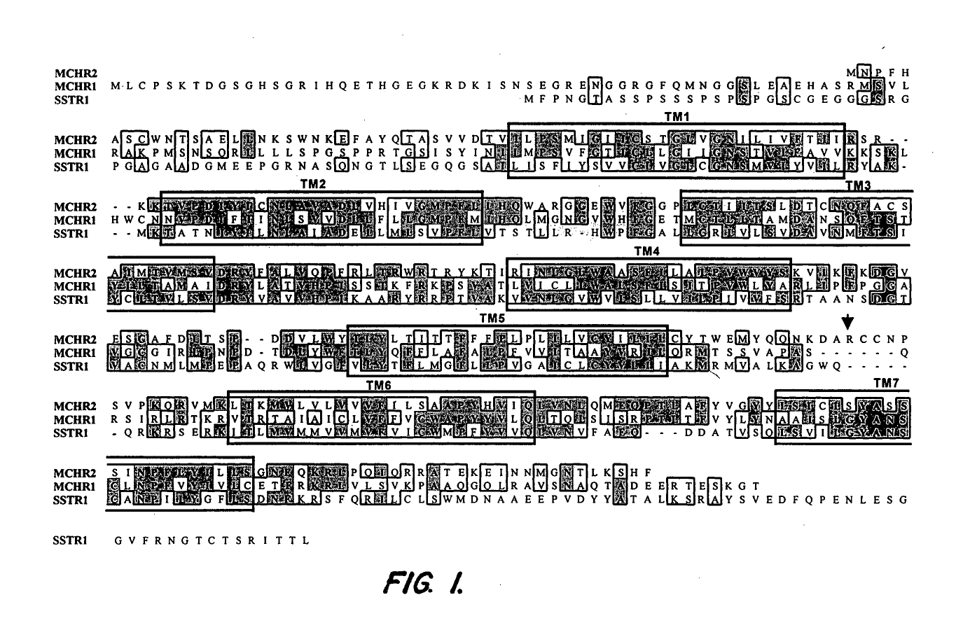

Sequence analysis of the protein sequence encoded by the full-length cDNA shows that it is closer to the MCH receptor than to the somatostatin receptor in evolution (see, Lee et al., Curr Opin. Pharmacol., “Orphan G Protein-Coupled Receptors in the CNS,” (2001) for a detailed discussion regarding somatostatin receptor-like orphan GPCRs). In FIG. 1, TGR342 is identified as MCHr2, the MCH receptor is identified as MCHr1, and the somatostatin receptor 1 is identified as SSTR1. Referring to FIG. 1, the shaded regions represent sequence identity, whereas the boxed regions represent similar amino acids. The transmembrane regions in FIG. 1 are labeled as TM1 through TM7.

Additionally, two representataive of an alternatively spliced TGR342 mRNA were identified by sequencing RACE products. This truncated alternatively spliced form has an extended fifth exon ending and lacks the sixth exon, giving rise to a predicted protein sequence that lacks the last two transmembrane domains.

TGR342 has 44% amino acid identity with MCH receptor protein GPRO_HUMAN (SWISS-PROT) in the transmembrane regions and an overall amino acid identity of 36%. The chromosomal location and genomic structure of TGR342 are different from those of the MCH receptor gene. TGR342, which has five coding exons and one non-coding exon, has been localized to the chromosome 6q21 region. The MCH receptor (MCHR1) gene lacks introns and is located on the chromosome 22q13.3. TGR342 has 31% amino acid identity to SSR5_HUMAN (SWISS-PROT) (somatostatin receptor 5) over 309 amino acids.

### Example 2

### Tissue Distribution of TGR342 mRNA

This example describes the procedures used to measure the tissue distribution pattern of TGR342 mRNA and the results obtained therefrom.

First, RT-PCR was performed. The primers used for PCR expression profiling were as follows:

TGR342Left primer
5′ GGAAAGTCCACGAACAATGAA 3′
TGR342Right primer
5′ TGAATAAGAAAAGGCATTCCAAC 3′

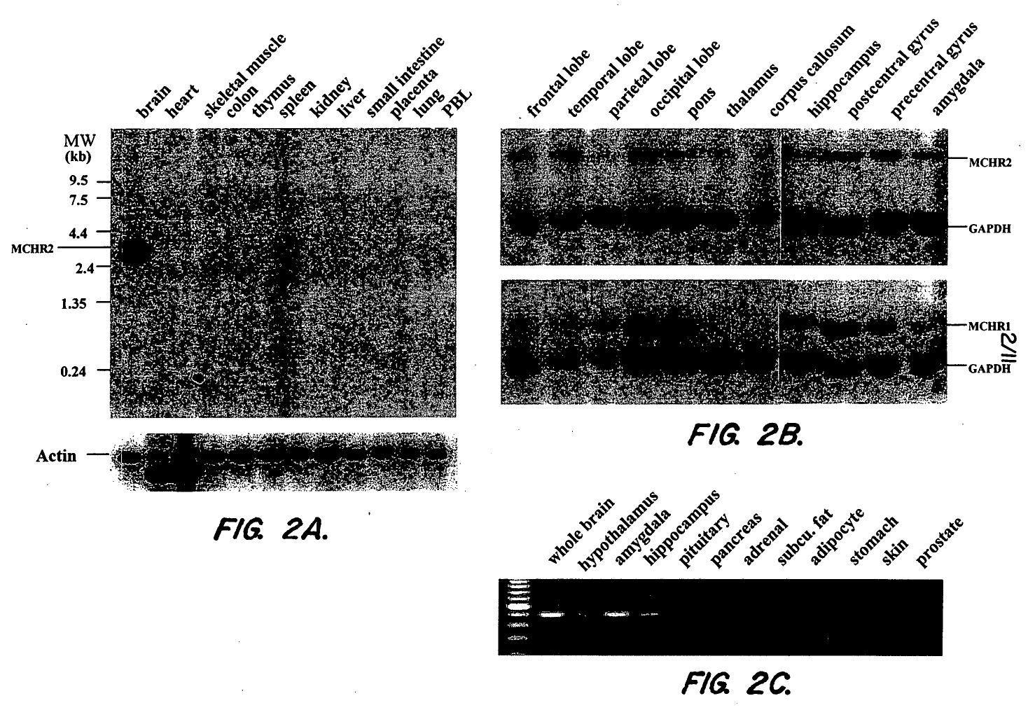

PCR analysis (e.g., FIG. 2C) shows that TGR342 mRNA was expressed in hippocampus, whole brain, amygdala and spleen tissue. Very weak expression was also observed in adipocytes and, with less stringent PCR conditions, in pituitary and pancreas.

In addition, Northern (Clontech 12-lane normal human tissue mRNA blot) and dot hybridization (Clontech multiple human tissue mRNA array blot) analyses were performed. The probe used for the northern was a 0.9 kb 3′RACE-PCR product. The sequences of the primers are as follows: Left primer: 5′CCAGTGTGGTAGATACAG TCATCCTCCCTTC 3′; Right primer (AP1 from Clontech): 5′ACTCACTATAGGGC TCGAGCGGC 3′.

Northern blot (e.g., FIG. 2A) and dot hybridization detected TGR342 expression only in human brain. On the Northern blot, a single species mRNA was detected in human brain with an estimated size of 2.5-3.0 kb. The dot hybridization data showed sub-regions of the brain showing significant hybridization signals include, in the order of signal intensity, paracentral gyrus of cerebral cortex, parietal lobe, accumbens nucleus, temporal lobe, frontal lobe, hippocampus, whole brain, fetal brain, occipital lobe, amygdala, caudate nucleus, cerebral Cortex, pons, putamen.

Expression in various regions of the brain (frontal lobe, temporal lobe, parietal lobe, occipital lobe, pons, thalamus, corpus callosum, hippocampus, postcentral gyrus, precentral gyrus, and amygdala) was also analyzed by northern blot (FIG. 2B). TGR342 expression was observed in each of these tissues.

In view of the determination that TGR342 (MCHR2) is expressed in particular regions of the brain, with varying intensities, TGR342 is believed to play a regulatory function in those regions.

### Example 3

### TGR342-Associated Signal Transduction

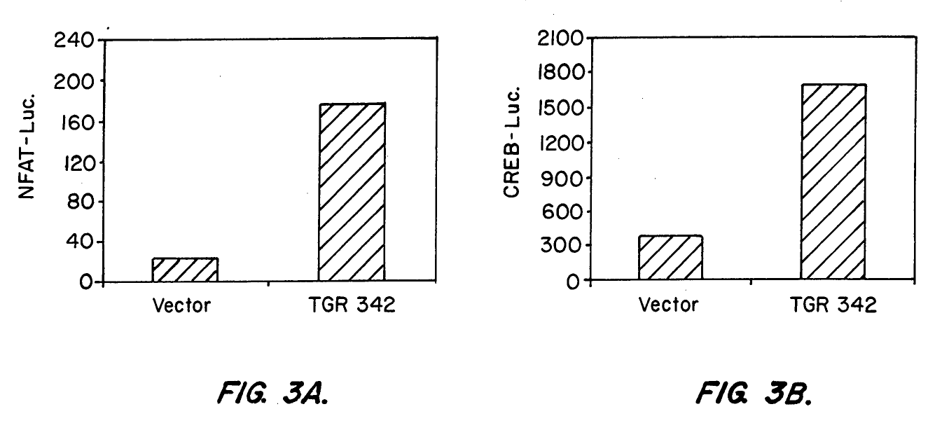

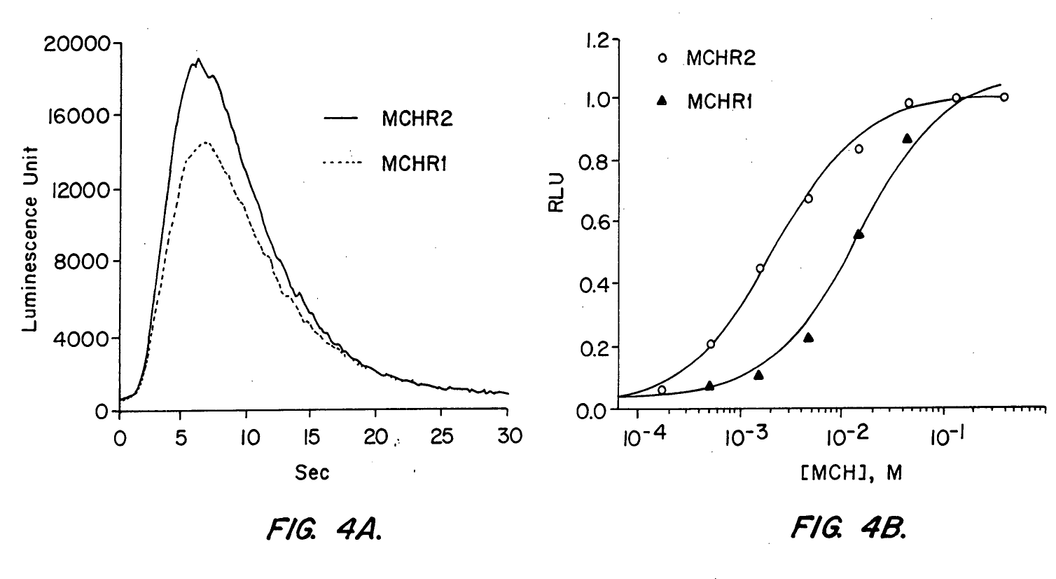

Cell-based assays were used to determine the signal transduction pathway of TGR342. The TGR342 cDNA was cloned into expression vector pEF6-V5 (Invitrogen) and introduced into CHO cells by co-transfection with reporter plasmid DNA pCREB-luc (luciferase trans-activating reporter) or pNFAT-luc using Lipofactamine 2000 (GIBCO BRL) following the protocol provided by the manufacturer. After 24 hours of incubation, the luciferase activity in the cells was assayed with the Dual Luciferase Assay kit from Promega following the instruction that came with the kit. Cells transfected with pNFAT/TGR342 showed activities in pCREB-luc and pNFAT-luc (FIG. 3). These results indicate that TGR342 can activate both PKA-CREB and Ca⁺⁺-NFAT pathways for its signal transduction. The Ca⁺⁺ signaling of TGR342 was confirmed by an aequorin assay as described below (FIG. 4).

### Example 4

### Identification of MCH as the Ligand of TGR342

A series of experiments were conducted in order to identify the natural ligand for TGR342.

First, an aequorin luminescence assay (Anal. Biochem., 272:34-42 (1999)), which measures receptor-mediated stimulation of intracellular calcium mobilization, was used to screen the Sigma-RBI biologically active peptide library. The library contains 60 different peptides, including MCH, the structurally similar peptide somatostatin, and the functional MCH antagonist α-MSH. HEK293 and CHO cells were transiently co-transfected with pEF6-V5/TGR342 and pcDNA3/AEQ (expressing aequorin). After 24 hours of incubation, the transfected cells were treated for one hour with serum free medium containing 2 μM coelenterazine f (Molecular Probes). The cells were then collected in the assay buffer HBSS (Hanks' Balanced Saline Solution containing 10 mM hepes and 0.1% BSA). Thereafter, the transfected cells were stimulated with the peptides at a concentration of 1 μM and the kinetics of fluorescence emission from the cells was recorded with a microplate luminometer (EG & G BERTHOLT). Of the 60 peptides, only MCH increased intracellular calcium levels in either HEK293 or CHO cells. Neither somatostatin nor α-MSH showed any detectable effect on the calcium levels. These results suggest that the MCH activates TGR342 and the activation is specific. The increase in intracellular levels of calcium upon stimulation with MCH also indicates that TGR342 is coupled to Gαq protein for signal transduction (see, Example 6). A dose response analysis in HEK293 cells transfected with only pEF6-V5 and pcDNA3/AEQ showed that that MCH activated TGR342 with an EC₅₀ of 2 nM, while MCH activated the MCH receptor with an EC₅₀ of about 10-12 nM (FIG. 4). The control plasmid pEF6-V5 did have any detectable effect. Thus, MCH induces similar dose-dependent stimulation of Ca²⁺ in cells transfected with either MCHR1 or TGR342 expression constructs.

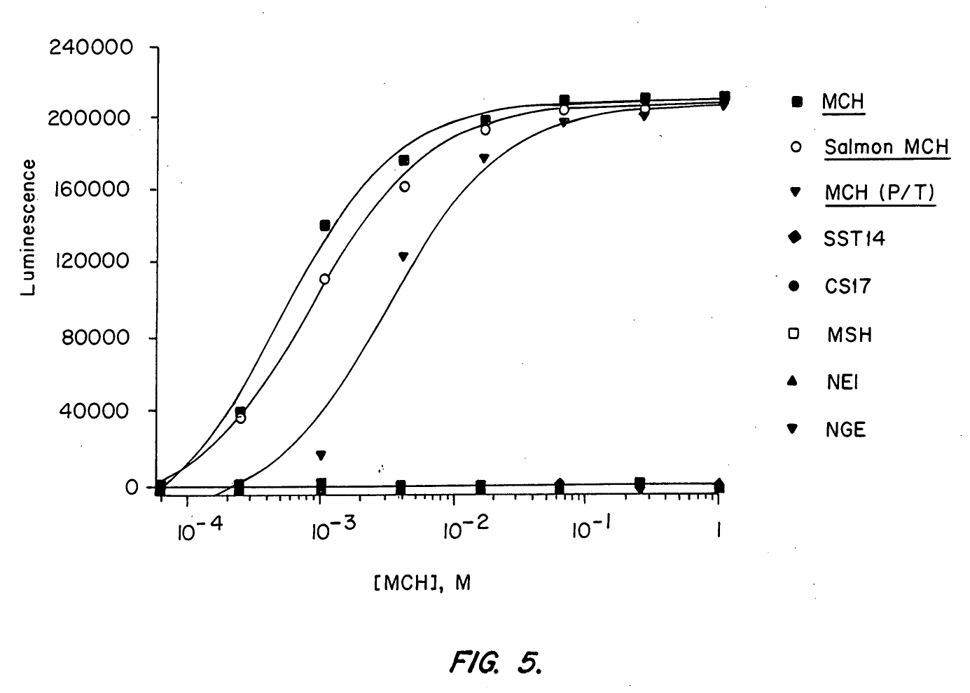

To further analyze the specificity of TGR342, several MCH-related peptides were tested for agonist activity. Salmon MCH has a high degree of homology to human MCH and activates MCHR1. It also activates TGR342 (FIG. 5). [Phe¹³, Tyr¹⁹]-MCH, a synthetic analog of MCH known to bind and activate MCHR1 also activates TGR342. Other peptides from the same MCH precursor neuropeptide-EI (NEI) and neuropeptide GE (NGE) were inactive. As MCHR1 and TGR342 share homology with somatostatin receptors, somatostatin-14, somatostatin-28, somatostatin analog, RC-160, and two peptides structurally very similar to somatostatin, cortistatin-14, and cortistatin-17, were also evaluated. None of these peptides exhibited activity at 1 μM. A functional antagonist of MCH, αMSH, was also inactive as an agonist at 1 μM (FIG. 5).

### Example 5

### Binding of MCH to TGR342

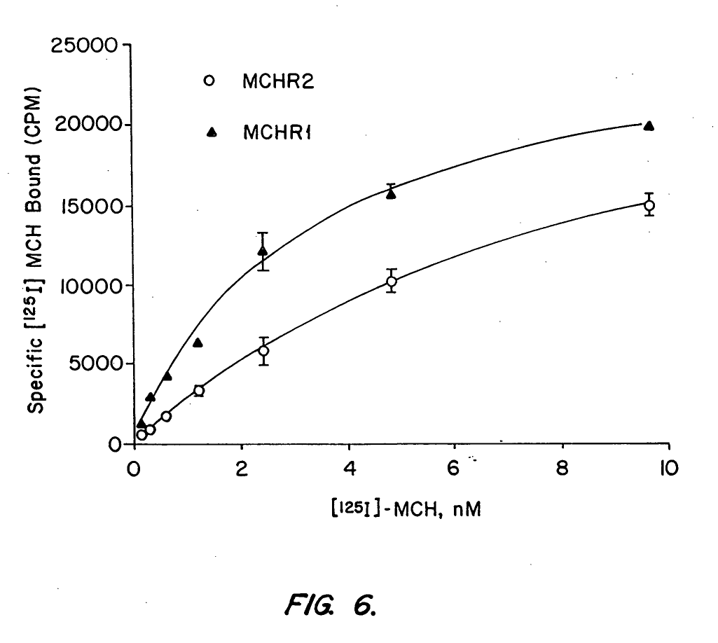

The ability of MCH to bind to TGR342 was also assessed. Membrane preparations from HEK293 cells that were transiently transfected with pEF6/V5-TGR342 were tested for the ability to bind [¹²⁵I]-MCH. Specific binding was calculated as the total binding subtracted by non-specific binding (membrane from mock-transfected cells.) As a control, [¹²⁵I]-MCH binding to membrane prepared from MCHR1-transfected cells was also measured in parallel. As shown in FIG. 6, MCH bound to TGR342 with an affinity (Kd=9.6 nM±0.5 nM) comparable to that of MCHR1 (Kd=3.1 nM±0.4 nM). The binding of MCH to TGR342 is specific, as it was inhibited by excess unlabeled MCH (data not shown). Based on the specific and high-affinity MCH binding that was observed for TGR342 and the functional assay results, TGR342 is a high affinity MCH receptor.

### Example 6

### TGR342 is Primarily Coupled to Gq

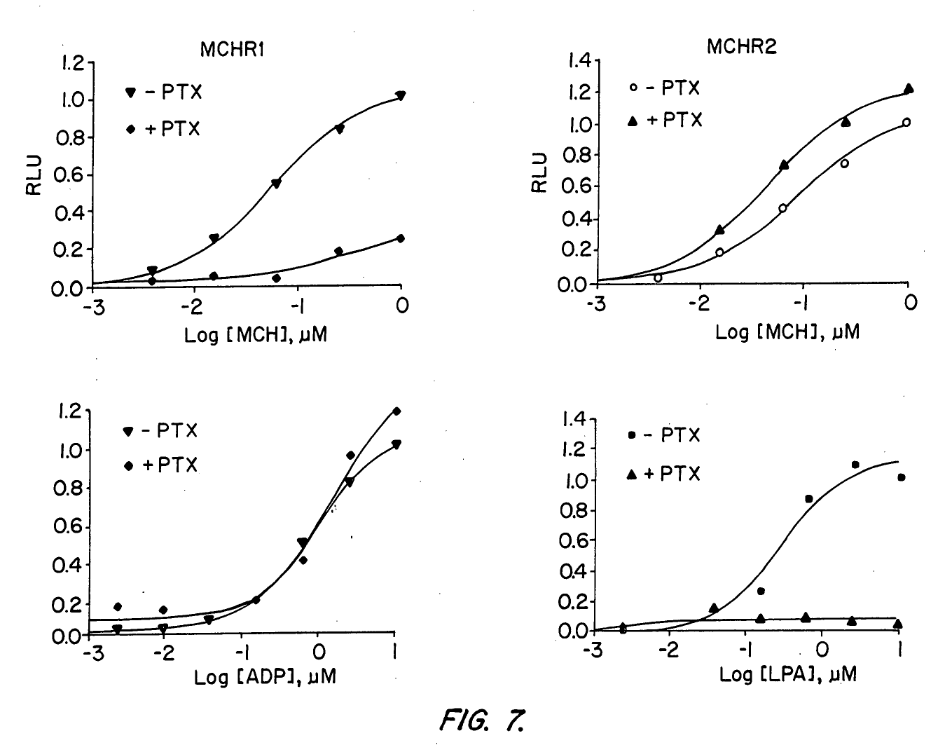

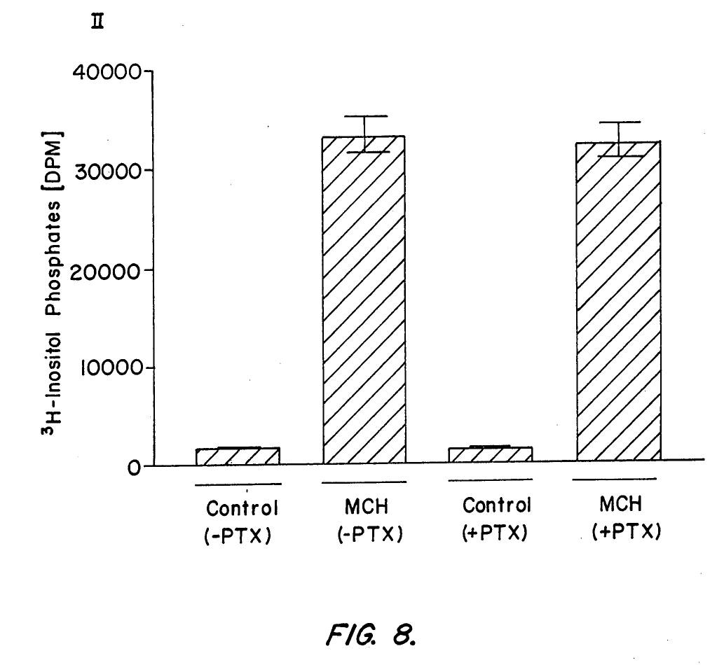

HEK293 cells transfected with TGR342 or MCHR1 were stimulated with MCH, ADP or PLA at indicated concentrations (FIGS. 7 and 8) with or without pretreatment with 100 ng/ml pertussis toxin (PTX) for 16 hr. The Ca²⁺ responses were determined with the aequorin assay. Relative luminescence is expressed as the fold-luminescence of that from the cells treated with the highest cocnetration of the ligand without PTX treatment. Cells were transfected with TGR342 or MCHR1 and stimulated with MCH.

In HEK293 (or CHO) cells transfected with pEF6/V5-TGR342, MCH stimulated increased levels of both inositol phosphates and Ca²⁺, and this effect was insensitive to PTX treatment (FIG. 7 and FIG. 8) In contrast, the Ca²⁺ response in cells transfected with MCHR1 was mostly inhibited, which is consistent with previous reports. LPA and ADP, which stimulate Ca²⁺ increase through endogenous Gi and Gq receptors, respectively (see, e.g., Moolenaar, Ann. N. Y Acad. Sci. 905:1-10, 2000; Fields et al., Biochem. J. 321:561-571, 1997), were tested as controls. As expected, LPA-induced Ca²⁺ response was PTX sensitive while the ADP-induced response was resistant (FIG. 7). These results demonstrate that TGR342 is primarily coupled to Gq.

### Example 7

### TGR 60 Nucleic Acid and Protein Sequences

Two novel TGR60 GPCRs were also identified. The cDNA sequence of the two cDNAs are provided in SEQ ID NO:7 and SEQ ID NO:9. The cDNAs were isolated using PCR with the following primers. TGR60L: Fwd, 5′-CACCATGCCAGCCAACTTCACAGAGGGCAGC-3′; Rev, 5′-CTAGATGAA TTCTGGCTTGGACAG-3′; TGR60S: Fwd, 5′-CACCATGCCAGCCAACTTCACAGA GGGCAGC-3′; Rev, 5′-CTAGTCATTTCCATCTATGATCCTGCA-3′.

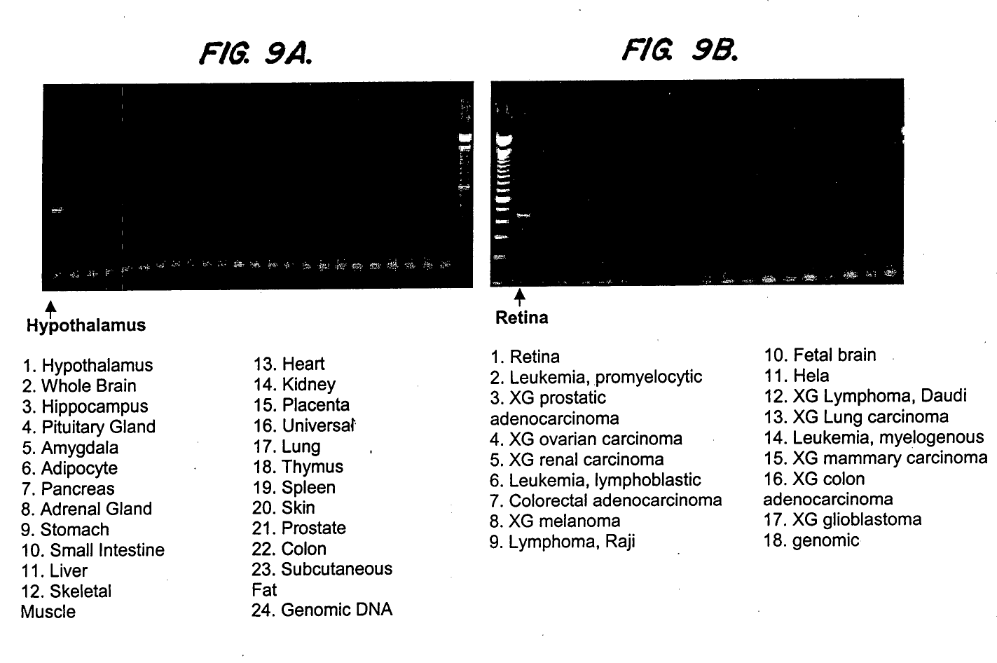

The two proteins are generated by alternative splicing of an mRNA. Expression of TGR60 was analyzed by PCR using the following primers:TGR60: Forward primer, 5′-CTGGAGCCTGTC TTTTCTGTTCTCC-3′; and Reverse primer, 5′-GGCAGGTTCTGAATGATCAC AGAGG-3′. The results show that TGR60 is expressed in the retina and hypothalamus (FIG. 9).

CG6111 (Celera fly genome project), which is available from GenBank under the accession number AAF56536, is the receptor for PDF in Drosophila. The present invention provides novel nucleic acid and protein CG6111 sequences (SEQ ID NO:11 and SEQ ID NO:12, respectively) that differ from the Celera fly genome project nucleic acid and protein sequences (SEQ ID NO:13 and SEQ ID NO:14, respectively).

The novel CG6111 nucleic acid sequence was identified by searching GenBank using the human TGR60 sequence as a query. A cDNA was isolated from adult flies and larva using PCR (Forward primer: 5′ATGAAATGTGACCACACTTTGTTC 3′; Reverse primer: 5′TGCCTTCACAGGATGTCCGTGTTC 3′). Sequence analysis of the Drosophila cDNA showed differences relative to the CG6 111 sequence set forth in the Celera fly genome project, which was derived from computer prediction of the sequence. The differences in the nucleic acid and protein sequences are indicated by large font, bolded, underlined characters in the sequences set forth in SEQ ID NOs:11, 12, 13, and 14. The DNA sequences differ by a single nucleotide at the indicated positions, and at their 3′ ends (see, SEQ ID NOs:11 and 13). The protein sequences differ at the carboxy terminus: the CG6111 protein sequence (SEQ ID NO:14) encoded by the nucleic acid sequence identified in the fly genome project includes the amino acid sequence RRGVSLKGNTDIL at the carboxy terminus, whereas the protein encoded by the cDNA has a V residue at the carboxy terminus (SEQ ID NO:12) instead of the RRGVSLKGNTDIL sequence

This difference in amino acid sequence can result in profound differences in activity of the protein. Accordingly, the novel fly nucleic acid and protein sequences provided herein can be used to identify modulators of GPCR activity and further, can be used as modulators of circadian rhythm, as well as a molecular tool for understanding the mechanism underlying circadian rhythm. Such modulators can also be used to modulate circadian rhythms in humans and other mammals. In addition, modulators of the fly nucleic acid and protein sequences of the invention can be used to target insect populations, for example, as pesticides.

### Example 8

### Two Additional GPCRs

Two GPCRs were identified by searching the public databases (Swiss-Prot and Genbank) and their nucleic acid sequences are provided in SEQ ID NO: 3 and 5. The deduced amino acid sequences are provided in SEQ ID NO:4 and 6. The novel GPCRs were designated TGR339 and TGR346.

TGR339 was identified from genomic sequences by searching the public databases. It is available under the accession number AC073957 (Genbank). It is 25% identical to CML2_HUMAN (SWISS-PROT) (chemokine like receptor 2) over 327 amino acids, and 23% idenitcal to IL8B_HUMAN (SWISS-PROT) (IL-8 receptor B) over 291 amino acids.

A 2.4 kb transcript has been detected in several human tissue including brain, kidney, liver, lung, placenta, adipose, spleen, lymph node, thymus, bone marrow, and fetal liver. The probe used for the northern is a 505 bp PCR product. The sequences of the primer are as follows:

TGR339Left primer
5′ ATCCCCTTCAATGTGTCCTC 3′
TGR339Right primer
5′ GCAGTAGCCCCAGGTAGTGT 3′

TGR346 was also identified from genomic sequences by searching the public databases. It is available under the accession number AC068256 (Genbank). It is 32% identical to NY2R_HUMAN (SWISS-PROT) (neuropeptide Y receptor 2) over 316 amino acids, and 31% identical to NP_{—}004876 (Genbank) (HLWAR77, receptor for NPAF and NPAFF neuropeptides) over 291 amino acids. The primers used for PCR expression profiling are as follows:

TGR346Left
5′ GCTTTCACAATGCTAGGTGAGG 3′
TGR346Right
5′ AGCAAGATGTCGTTTGAGCTTT 3′

It is noted that several alternative splice forms for TFR346 were identified. For instance, three ESTs for TGR346 have been identified in the public databases that are non-functional spliced variants.

Two novel mouse TGR346 nucleic acid sequences were also identified. These sequences were first identified by using the human sequence to search the mouse genome. Two genes were identified and the cDNAs subsequently isolated from mouse brain using PCR. The two mouse proteins share 73% identity. The protein mTGR346a is 83% identical to human-TGR346 and mTGR346b is 77% identical to human TGR346.

Primers for FLcloning:

ms346a

| | |
|---|---|
| Fwd: | 5′-CACCATGCAGGCGCTCAACATCACCGC-3′ |
| Rev: | 5′-TTACAGTTCATGTCCACTGCCGAAAGTA-3′ |

ms346b

| | |
|---|---|
| Fwd: | 5′-CACCATGTCGTGGAACTTGACCGCGGA-3′ |
| Rev: | 5′-CTAAAGAGGACAAGATGCCACTTTTGA-3′ |

Primers for RACE:

ms346a

| | |
|---|---|
| RACE1 | 5′-GCTCTTTGGCAACTCTCTGGTCATC-3′ |
| RACE2 | 5′ GCACGTACAACGCCTCGAGATTAAG-3′ |
| RACE3 | 5′-ACCTTCATCCTCGTCATCCTCTTCC-3′ |

ms346b

| | |
|---|---|
| RACE1 | 5′-ACGCCCTGGTAGTCTATGTGGTGAC-3′ |
| RACE2 | 5′-TGCACCAGAAGATCTACACCACCTTC-3′ |
| RACE3 | 5′-ATTCTTGGCACCCTCTTCCTGCTAC-3′ |

Expression of the two genes was analyzed by QPCR. Primers for analysis of ms346a expression were: Fwd, 5′AAGGCAACTCAAGCGACAGC 3′ and Rev, 5′CAAATGA TATTAGCTATGAGGATATCATTACA. The probe was 6FAM-CTGAAAACTCTACTTTCGGCAGTGGACATGA-TAMRA. The QPCR primers to analyze ms346b expression were: Fwd, 5′TCTTGTCCTCTTTAGTTCCGAATTTC 3′; Rev, 5′TTCGATTACAGTATGACAGATACTCATTCT 3′. The probe was 6FAM-CTCTGCTGTAGACGTGAACACTGTACCAATGTC-TAMRA

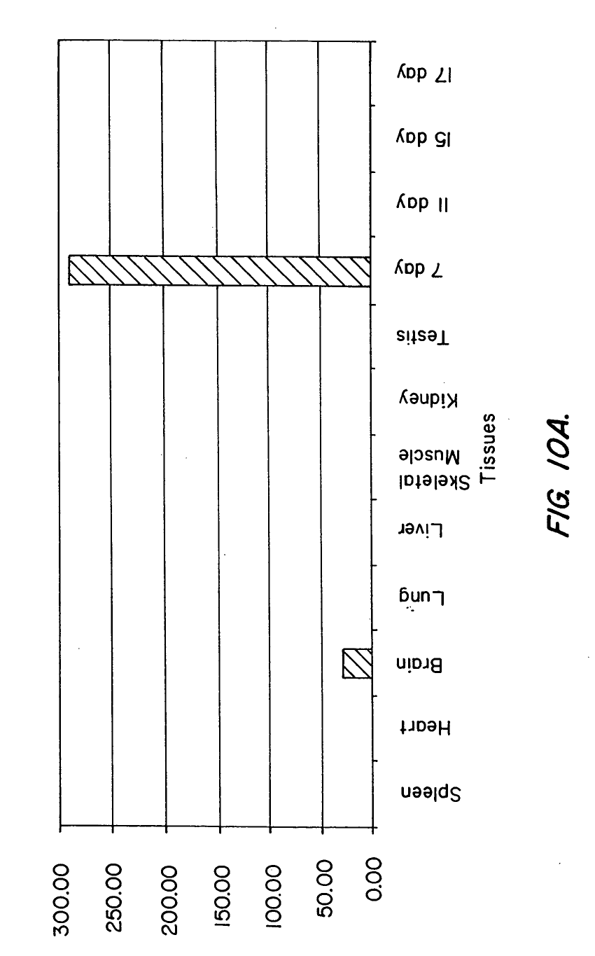

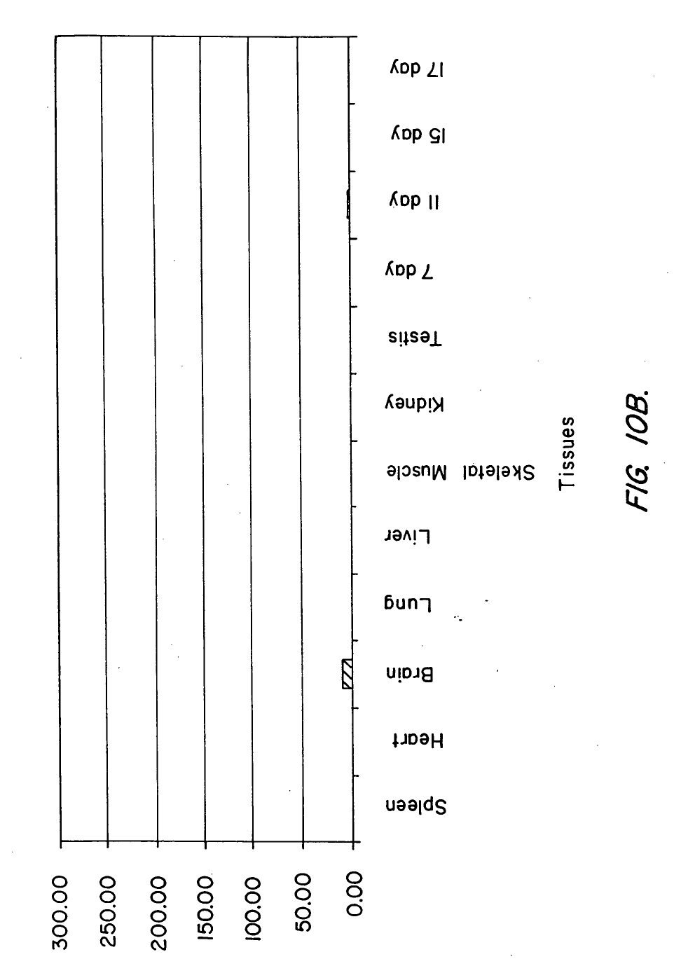

The results show that 346a is expressed in the brain. The m346a RNA is also expressed at embryonic day seven (FIG. 10A). The m346b gene is also expressed in the brain, with a small amount of expression also observed at embryonic day 11 (FIG. 10B).

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

Table of Receptor Nucleic Acid and Protein Sequences

SEQ ID NO:1
Human TGR342 Nucleic Acid
(5 exons)
ATGAATCCATTTCATGCATCTTGTTGGAACACCTCTGCCGAACTTTTAAACAAATCCTGGAATAA
AGAGTTTGCTTATCAAACTGCCAGTGTGGTAGATACAGTCATCCTCCCTTCCATGATTGGGATTA
TCTGTTCAACAGGGCTGGTTGGCAACATCCTCATTGTATTCACTATAATAAGATCCAGGAAAAAA
ACAGTCCCTGACATCTATATCTGCAACCTGGCTGTGGCTGATTTGGTCCACATAGTTGGAATGCC
TTTTCTTATTCACCAATGGGCCCGAGGGGGAGAGTGGGTGTTTGGGGGGCCTCTCTGCACCATCA
TCACATCCCTGGATACTTGTAACCAATTTGCCTGTAGTGCCATCATGACTGTAATGAGTGTGGAC
AGGTACTTTGCCCTCGTCCAACCATTTCGACTGACACGTTGGAGAACAAGGTACAAGACCATCCG
GATCAATTTGGGCCTTTGGGCAGCTTCCTTTATCCTGGCATTGCCTGTCTGGGTCTACTCGAAGG
TCATCAAATTTAAAGACGGTGTTGAGAGTTGTGCTTTTGATTTGACATCCCCTGACGATGTACTC
TGGTATACACTTTATTTGACGATAACAACTTTTTTTTTCCCTCTACCCTTGATTTTGGTGTGCTA
TATTTTAATTTTATGCTATACTTGGGAGATGTATCAACAGAATAAGGATGCCAGATGCTGCAATC
CCAGTGTACCAAAACAGAGAGTGATGAAGTTGACAAAGATGGTGCTGGTGCTGGTGGTAGTCTTT
ATCCTGAGTGCTGCCCCTTATCATGTGATACAACTGGTGAACTTACAGATGGAACAGCCCACACT
GGCCTTCTATGTGGGTTATTACCTCTCCATCTGTCTCAGCTATGCCAGCAGCAGCATTAACCCTT
TTCTCTACATCCTGCTGAGTGGAAATTTCCAGAAACGTCTGCCTCAAATCCAAAGAAGAGCGACT
GAGAAGGAAATCAACAATATGGGAAACACTCTGAAATCACACTTTTAG
SEQ ID NO:2
Human TGR342 Protein
MNPFHASCWNTSAELLNKSWNKEFAYQTASVVDTVILPSMIGIICSTGLVGNILIVFTIIRSRKK
TVPDIYICNLAVADLVHIVGMPFLIHQWARGGEWVFGGPLCTIITSLDTCNQFACSAIMTVMSVD
RYFALVQPFRLTRWRTRYKTIRINLGLWAASFILALPVWVYSKVIKFKDGVESCAFDLTSPDDVL
WYTLYLTITTFFFPLPLILVCYILILCYTWEMYQQNKDARCCNPSVPKQRVMKLTKMVLVLVVVF
ILSAAPYHVIQLVNLQMEQPTLAFYVGYYLSICLSYASSSINPFLYILLSGNFQKRLPQIQRRAT
EKEINNMGNTLKSHF*
SEQ ID NO:3
Human TGR339 Nucleic Acid
(single exon)
ATGTGGAGCTGCAGCTGGTTCAACGGCACAGGGCTGGTGGAGGAGCTGCCTGCCTGCCAGGACCT
GCAGCTGGGGCTGTCACTGTTGTCGCTGCTGGGCCTGGTGGTGGGCGTGCCAGTGGGCCTGTGCT
ACAACGCCCTGCTGGTGCTGGCCAACCTACACAGCAAGGCCAGCATGACCATGCCGGACGTGTAC
TTTGTCAACATGGCAGTGGCAGGCCTGGTGCTCAGCGCCCTGGCCCCTGTGCACCTGCTCGGCCC
CCCGAGCTCCCGGTGGGCGCTGTGGAGTGTGGGCGGCGAAGTCCACGTGGCACTGCAGATCCCCT
TCAATGTGTCCTCACTGGTGGCCATGTACTCCACCGCCCTGCTGAGCCTCGACCACTACATCGAG
CGTGCACTGCCGCGGACCTACATGGCCAGCGTGTACAACACGCGGCACGTGTGCGGCTTCGTGTG
GGGTGGCGCGCTGCTGACCAGCTTCTCCTCGCTGCTCTTCTACATCTGCAGCCATGTGTCCACCC
GCGCGCTAGAGTGCGCCAAGATGCAGAACGCAGAAGCTGCCGACGCCACGCTGGTGTTCATCGGC
TACGTGGTGCCAGCACTGGCCACCCTCTACGCGCTGGTGCTACTCTCCCGCGTCCGCAGGGAGGA
CACGCCCCTGGACCGGGACACGGGCCGGCTGGAGCCCTCGGCACACAGGCTGCTGGTGGCCACCG
TGTGCACGCAGTTTGGGCTCTGGACGCCACACTATCTGATCCTGCTGGGGCACACGGTCATCATC
TCGCGAGGGAAGCCCGTGGACGCACACTACCTGGGGCTACTGCACTTTGTGAAGGATTTCTCCAA
ACTCCTGGCCTTCTCCAGCAGCTTTGTGACACCACTTCTCTACCGCTACATGAACCAGAGCTTCC
CCAGCAAGCTCCAACGGCTGATGAAAAAGCTGCCCTGCGGGGACCGGCACTGCTCCCCGGACCAC
ATGGGGGTGCAGCAGGTGCTGGCGTAG
SEQ ID NO:4
Human TGR339 Protein
MWSCSWFNGTGLVEELPACQDLQLGLSLLSLLGLVVGVPVGLCYNALLVLANLHSKASMTMPDVY
FVNMAVAGLVLSALAPVHLLGPPSSRWALWSVGGEVHVALQIPFNVSSLVAMYSTALLSLDHYIE
RALPRTYMASVYNTRHVCGFVWGGALLTSFSSLLFYICSHVSTRALECAKMQNAEAADATLVFIG
YVVPALATLYALVLLSRVRREDTPLDRDTGRLEPSAHRLLVATVCTQFGLWTPHYLILLGHTVII
SRGKPVDAHYLGLLHFVKDFSKLLAFSSSFVTPLLYRYMNQSFPSKLQRLMKKLPCGDRHCSPDH
MGVQQVLA*
SEQ ID NO:5
Human TGR346 Nucleic Acid
(5 exons)
ATGCAGGCGCTTAACATTACCCCGGAGCAGTTCTCTCGGCTGCTGCGGGACCACAACCTGACGCG
GGAGCAGTTCATCGCTCTGTACCGGCTGCGACCGCTCGTCTACACCCCAGAGCTGCCGGGACGCG
CCAAGCTGGCCCTCGTGCTCACCGGCGTGCTCATCTTCGCCCTGGCGCTCTTTGGCAATGCTCTG
GTGTTCTACGTGGTGACCCGCAGCAAGGCCATGCGCACCGTCACCAACATCTTTATCTGCTCCTT
GGCGCTCAGTGACCTGCTCATCACCTTCTTCTGCATTCCCGTCACCATGCTCCAGAACATTTCCG
ACAACTGGCTGGGGGGTGCTTTCATTTGCAAGATGGTGCCATTTGTCCAGTCTACCGCTGTTGTG
ACAGAAATCCTCACTATGACCTGCATTGCTGTGGAAAGGCACCAGGGACTTGTGCATCCTTTTAA
AATGAAGTGGCAATACACCAACCGAAGGGCTTTCACAATGCTAGGTGTGGTCTGGCTGGTGGCAG
TCATCGTAGGATCACCCATGTGGCACGTGCAACAACTTGAGATCAAATATGACTTCCTATATGAA
AAGGAACACATCTGCTGCTTAGAAGAGTGGACCAGCCCTGTGCACCAGAAGATCTACACCACCTT
CATCCTTGTCATCCTCTTCCTCCTGCCTCTTATGGTGATGCTTATTCTGTACAGTAAAATTGGTT
ATGAACTTTGGATAAAGAAAAGAGTTGGGGATGGTTCAGTGCTTCGAACTATTCATGGAAAAGAA
ATGTCCAAAATAGCCAGGAAGAAGAAACGAGCTGTCATTATGATGGTGACAGTGGTGGCTCTCTT
TGCTGTGTGCTGGGCACCATTCCATGTTGTCCATATGATGATTGAATACAGTAATTTTGAAAAGG
AATATGATGATGTCACAATCAAGATGATTTTTGCTATCGTGCAAATTATTGGATTTTCCAACTCC
ATCTGTAATCCCATTGTCTATGCATTTATGAATGAAAACTTCAAAAAAAATGTTTTGTCTGCAGT
TTGTTATTGCATAGTAAATAAAACCTTCTCTCCAGCACAAAGGCATGGAAATTCAGGAATTACAA
TGATGCGGAAGAAAGCAAAGTTTTCCCTCAGAGAGAATCCAGTGGAGGAAACCAAAGGAGAAGCA
TTCAGTGATGGCAACATTGAAGTCAAATTGTGTGAACAGACAGAGGAGAAGAAAAAGCTCAAACG
ACATCTTGCTCTCTTTAGGTCTGAACTGGCTGAGAATTCTCCTTTAGACAGTGGGCATTAA
SEQ ID NO:6
Human TGR346 Protein
MQALNITPEQFSRLLRDHNLTREQFIALYRLRPLVYTPELPGRAKLALVLTGVLIFALALFGNAL
VFYVVTRSKAMRTVTNIFICSLALSDLLITFFCIPVTMLQNISDNWLGGAFICKMVPFVQSTAVV
TEILTMTCIAVERHQGLVHPFKMKWQYTNRRAFTMLGVVWLVAVIVGSPMWHVQQLEIKYDFLYE
KEHICCLEEWTSPVHQKIYTTFILVILFLLPLMVMLILYSKIGYELWIKKRVGDGSVLRTIHGKE
MSKIARKKKRAVIMMVTVVALFAVCWAPFHVVHMMIEYSNFEKEYDDVTIKMIFAIVQIIGFSNS
ICNPIVYAFMNENFKKNVLSAVCYCIVNKTFSPAQRHGNSGITMMRKKAKFSLRENPVEETKGEA
FSDGNIEVKLCEQTEEKKKLKRHLALFRSELAENSPLDSGH*
SEQ ID NO:7
Human TGR60 Nucleic Acid Sequence 1
Alternative splicing in the last coding exon gives rise to two
cDNA species. Diverged sequences (compared to SEQ ID NO:9) are
underlined.
GCTGCCCAGCTCTCAGGAGGCAAGCTGGACTCCCTCACTCGGCTGCAGGAGCAAGGACAGTGAGG
CTCAACCCCGCCTGAGCCATGCCAGCCAACTTCACAGAGGGCAGCTTCGATTCCAGTGGGACCGG
GCAGACGCTGGATTCTTCCCCAGTGGCTTGCACTGAAACAGTGACTTTTACTGAAGTGGTGGAAG
GAAAGGAATGGGGTTCCTTCTACTACTCCTTTAAGACTGAGCAATTGATAACTCTGTGGGTCCTC
TTTGTTTTTACCATTGTTGGAAACTCCGTTGTGCTTTTTTCCACATGGAGGAGAAAGAAGAAGTC
AAGAATGACCTTCTTTGTGACTCAGCTGGCCATCACAGATTCTTTCACAGGACTGGTCAACATCT
TGACAGATATTAATTGGCGATTCACTGGAGACTTCACGGGACCTGACCTGGTTTGCCGAGTGGTC
CGCTATTTGCAGGTTGTGCTGCTCTACGCCTCTACCTACGTCCTGGTGTCCCTCAGCATAGACAG
ATACCATGCCATCGTCTACCCCATGAAGTTCCTTCAAGGAGAAAAGCAAGCCAGGGTCCTCATTG
TGATCGCCTGGAGCCTGTCTTTTCTGTTCTCCATTCCCACCCTGATCATATTTGGGAAGAGGACA
CTGTCCAACGGTGAAGTGCAGTGCTGGGCCCTGTGGCCTGACGACTCCTACTGGACCCCATACAT
GACCATCGTGGCCTTCCTGGTGTACTTCATCCCTCTGACAATCATCAGCATCATGTATGGCATTG
TGATCCGAACTATTTGGATTAAAAGCAAAACCTACGAAACAGTGATTTCCAACTGCTCAGATGGG
AAACTGTGCAGCAGCTATAACCGAGGACTCATCTCAAAGGCAAAAATCAAGGCTATCAAGTATAG
CATCATCATCATTCTTGCCTTCATCTGCTGTTGGAGTCCATACTTCCTGTTTGACATTTTGGACA
ATTTCAACCTCCTTCCAGACACCCAGGAGCGTTTCTATGCCTCTGTGATCATTCAGAACCTGCCA
GCATTGAATAGTGCCATCAACCCCCTCATCTACTGTGTCTTCAGCAGCTCCATCTCTTTCCCCTG
CAGGGAGCAAAGATCACAGGATTCCAGAATGACGTTCCGGGAGAGAACTGAGAGGCATGAGATGC
AGATTCTGTCCAAGCCAGAATTCATCTAGACCCTAGGGCAGTGCCAGTGCTAGGCTGAGCACCAT
CAGCTCTTCCAGGTCCTTGTCACCTGCTTGGGCACGTGCATGGAACCCGAGCCAACTTCACC
SEQ ID NO:8
Human TGR60 Protein Sequence 1
Alternative splicing in the last coding exon gives rise to two
cDNA species that encode two proteins. Diverged sequences
(compared to SEQ ID NO:10) are underlined.
MPANFTEGSFDSSGTGQTLDSSPVACTETVTFTEVVEGKEWGSFYYSFKTEQLITLWVLFVFTIV
GNSVVLFSTWRRKKKSRMTFFVTQLAITDSFTGLVNILTDINWRFTGDFTGPDLVCRVVRYLQVV
LLYASTYVLVSLSIDRYHAIVYPMKFLQGEKQARVLIVIAWSLSFLFSIPTLIIFGKRTLSNGEV
QCWALWPDDSYWTPYMTIVAFLVYFIPLTIISIMYGIVIRTIWIKSKTYETVISNCSDGKLCSSY
NRGLISKAKIKAIKYSIIIILAFICCWSPYFLFDILDNFNLLPDTQERFYASVIIQNLPALNSAI
NPLIYCVFSSSISFPCREQRSQDSRMTFRERTERHEMQILSKPEFI
SEQ ID NO:9
Human TGR60 Nucleic Acid Sequence 2
Alternative splicing in the last coding exon gives rise to two
cDNA species. Diverged sequences (compared to SEQ ID NO:7) are
underlined.
GCTGCCCAGCTCTCAGGAGGCAAGCTGGACTCCCTCACTCGGCTGCAGGAGCAAGGACAGTGAGG
CTCAACCCCGCCTGAGCCATGCCAGCCAACTTCACAGAGGGCAGCTTCGATTCCAGTGGGACCGG
GCAGACGCTGGATTCTTCCCCAGTGGCTTGCACTGAAACAGTGACTTTTACTGAAGTGGTGGAAG
GAAAGGAATGGGGTTCCTTCTACTACTCCTTTAAGACTGAGCAATTGATAACTCTGTGGGTCCTC
TTTGTTTTTACCATTGTTGGAAACTCCGTTGTGCTTTTTTCCACATGGAGGAGAAAGAAGAAGTC
AAGAATGACCTTCTTTGTGACTCAGCTGGCCATCACAGATTCTTTCACAGGACTGGTCAACATCT
TGACAGATATTAATTGGCGATTCACTGGAGACTTCACGGGACCTGACCTGGTTTGCCGAGTGGTC
CGCTATTTGCAGGTTGTGCTGCTCTACGCCTCTACCTACGTCCTGGTGTCCCTCAGCATAGACAG
ATACCATGCCATCGTCTACCCCATGAAGTTCCTTCAAGGAGAAAAGCAAGCCAGGGTCCTCATTG
TGATCGCCTGGAGCCTGTCTTTTCTGTTCTCCATTCCCACCCTGATCATATTTGGGAAGAGGACA
CTGTCCAACGGTGAAGTGCAGTGCTGGGCCCTGTGGCCTGACGACTCCTACTGGACCCCATACAT
GACCATCGTGGCCTTCCTGGTGTACTTCATCCCTCTGACAATCATCAGCATCATGTATGGCATTG
TGATCCGAACTATTTGGATTAAAAGCAAAACCTACGAAACAGTGATTTCCAACTGCTCAGATGGG
AAACTGTGCAGCAGCTATAACCGAGGACTCATCTCAAAGGCAAAAATCAAGGCTATCAAGTATAG
CATCATCATCATTCTTGCCTTCATCTGCTGTTGGAGTCCATACTTCCTGTTTGACATTTTGGACA
ATTTCAACCTCCTTCCAGACACCCAGGAGCGTTTCTATGCCTCTGTGATCATTCAGAACCTGCCA
GCATTGAATAGTGCCATCAACCCCCTCATCTACTGTGTCTTCAGCAGCTCCATCTCTTTCCCCTG
CAGGATCATAGATGGAAATGACTAGCCTTGTCTCAGATGACACTTCGAACTTTGGACTTTTGAGT
TAATGTTGGAATAAGTTAAGACTTTCGGGGACTGTTGGGAAGGCAGGATTGTATTTTGAAATTTG
AGAAGGACATAAAATTTGGGAGGGGGCAGCATGGAATCATATGGTCTAGATATATGACCCTGTCC
AAATCTCAAATCTAACTGTAATTCCCAGTGTTGGAGGTGGGGTCTGGTGGGAGGTGATTTGATCA
TGGAGGTGGAGTTCTCATTAATGATTTAGAGCCATCCCTTTTGTTATGGTATAGTGAGTGAGTTA
TCACAAGATCTCTTTGTTTAAAAGTTTGTGGTACCTCCCACCTCTCTCTCTTGCTCCTGCTCTGG
CCATGTAAGACGTGCCTGCTTCCCCTTCACCTTCTTGCATGATTGTAAGTTTCCTGAGGCCTCCC
CAAAAGCAGAAGCCACTATGCTTCCTGAACAGCCAATGGAACCATGAGCCAATTAAACCTCTTCT
TTA
SEQ ID NO:10
Human TGR60 Protein Sequence 2
Alternative splicing in the last coding exon gives rise to two
cDNA species that encode two proteins. Diverged sequences
(compared to SEQ ID NO:8) are underlined.
MPANFTEGSFDSSGTGQTLDSSPVACTETVTFTEVVEGKEWGSFYYSFKTEQLITLWVLFVFTIV
GNSVVLFSTWRRKKKSRMTFFVTQLAITDSFTGLVNILTDINWRFTGDFTGPDLVCRVVRYLQVV
LLYASTYVLVSLSIDRYHAIVYPMKFLQGEKQARVLIVIAWSLSFLFSIPTLIIFGKRTLSNGEV
QCWALWPDDSYWTPYMTIVAFLVYFIPLTIISIMYGIVIRTIWIKSKTYETVISNCSDGKLCSSY
NRGLISKAKIKAIKYSIIIILAFICCWSPYFLFDILDNFNLLPDTQERFYASVIIQNLPALNSAI
NPLIYCVFSSSISFPCRIIDGND
SEQ ID NO:11
Drosophila CG6111 Nucleic Acid Sequence
ATGAAATGTGACCACACTTTGTTCTTTGCACTCTTTCAGACCGAACAGTTTGCTGTGCTCTGGAT
CCTGTTCACCGTCATCGTTCTGGGCAATTCAGCTGTTCTGTTCGTGATGTTCATCAACAAGAATC
GCAAGTCGCGGATGAACTACTTCATTAAACAGCTGGCATTGGCAGATCTGTGCGTGGGACTGCTC
AACGTCCTCACCGACATCATATGGCGCATCACGATTTCGTGGCGGGCAGGCAACCTGGCCTGCAA
GGCCATCCGCTTCTCGCAGGTCTGCGTCACATACTCGTCCACCTACGTGCTGGTGGCCATGAGCA
TCGACAGATACGATGCCATCACACACCCCATGAACTTCTCAAAGTCGTGGAAAAGAGCCCGTCAC
CTGGTGGCTGGCGCATGGCTCATCTCGGCGTTGTTTTCGCTTCCCATCCTGGTTTTGTACGAGGA
GAAGCTCATCCAAGGACATCCGCAATGCTGGATTGAGTTGGGTTCACCGATCGCCTGGCAGGTAT
ACATGAGCCTGGTGTCGGCCACTCTATTTGCCATTCCTGCGCTGATCATATCTGCCTGCTATGCG
ATCATCGTAAAGACGATTTGGGCAAAGGGTTCCATTTTTGTACCCACGGAACGTGCTGGTTTTGG
AGCTGCACCTGCCAGGAGGGCCAGCTCGAGGGGCATTATTCCACGGGCAAAGGTCAAAACGGTCA
AGATGACATTGACCATCGTGTTTGTGTTCATCATCTGCTGGTCGCCGTATATCATCTTCGATCTG
CTGCAGGTCTTTGGCCAGATTCCACACTCACAGACCAACATTGCCATCGCCACCTTCATCCAAAG
TCTGGCACCGCTGAACTCGGCGGCGAATCCACTAATCTATTGCCTCTTCTCATCGCAGGTCTTTC
GCACATTAAGTCGCTTTCCGCCTTTTAAGTGGTTCACATGCTGCTGCAAGTCATACCGCAACAAC
TCGCAGCAAAACCGCTGCCACACGGTTGGTCGTCGGCTTCACAACAGTTGCGATTCGATGAGGAC
ACTGACCACTTCGTTGACGGTTTCCCGAAGGTCCACCAACAAGACGAACGCCCGTGTGGTAATCT
GCGAACGTCCCACCAAGGTGGTTACCGTGCCAGCCATGTCGGAGGTATGA
SEQ ID NO:12
Drosophila CG6111 Protein Sequence
MKCDHTLFFALFQTEQFAVLWILFTVIVLGNSAVLFVMFINKNRKSRMNYFIKQLALADLCVGLL
NVLTDIIWRITISWRAGNLACKAIRFSQVCVTYSSTYVLVAMSIDRYDAITHPMNFSKSWKRARH
LVAGAWLISALFSLPILVLYEEKLIQGHPQCWIELGSPIAWQVYMSLVSATLFAIPALIISACYA
IIVKTIWAKGSIFVPTERAGFGAAPARRASSRGIIPRAKVKTVKMTLTIVFVFIICWSPYIIFDL
LQVFGQIPHSQTNIAIATFIQSLAPLNSAANPLIYCLFSSQVFRTLSRFPPFKWFTCCCKSYRNN
SQQNRCHTVGRRLHNSCDSMRTLTTSLTVSRRSTNKTNARVVICERPTKVVTVPAMSEV
SEQ ID NO:13
CG6111 Celera fly genome project DNA sequence
ATGAAATGTGACCACACTTTGTTCTTTGCACTCTTCCAGACCGAACAGTTTGCTGTGCTCTGGAT
CCTGTTCACCGTCATCGTTCTGGGCAATTCAGCTGTTCTGTTCGTGATGTTCATCAACAAGAATC
GCAAGTCGCGGATGAACTACTTCATTAAACAGCTGGCATTGGCAGATCTGTGCGTGGGACTGCTC
AACGTCCTCACCGACATCATATGGCGCATCACGATTTCGTGGCGGGCAGGCAACCTGGCCTGCAA
GGCCATCCGCTTCTCGCAGGTCTGCGTCACATACTCGTCCACCTACGTGCTGGTGGCCATGAGCA
TCGACAGATACGATGCCATCACACACCCCATGAACTTCTCAAAGTCGTGGAAAAGAGCCCGTCAC
CTGGTGGCTGGCGCATGGCTCATCTCGGCGTTGTTTTCGCTTCCCATCCTGGTTTTGTACGAGGA
GAAGCTCATCCAAGGACATCCGCAATGCTGGATTGAGTTGGGTTCACCGATCGCCTGGCAGGTGT
ACATGAGCCTGGTGTCGGCCACTCTATTTGCCATTCCTGCGCTGATCATATCTGCCTGCTATGCG
ATCATCGTAAAGACGATTTGGGCAAAGGGTTCCATTTTTGTACCCACGGAACGTGCTGGTTTTGG
AGCTGCACCTGCCAGGAGGGCCAGCTCGAGGGGCATTATTCCACGGGCAAAGGTCAAAACGGTCA
AGATGACATTGACCATCGTGTTTGTGTTCATCATCTGCTGGTCGCCGTATATCATCTTCGATCTG
CTGCAGGTCTTTGGCCAGATTCCACACTCACAGACCAACATTGCCATCGCCACCTTCATCCAAAG
TCTGGCACCGCTGAACTCGGCGGCGAATCCACTAATCTATTGCCTCTTCTCATCGCAGGTCTTTC
GCACATTAAGTCGCTTTCCGCCTTTTAAGTGGTTCACATGCTGCTGCAAGTCATACCGCAACAAC
TCGCAGCAAAACCGCTGCCACACGGTTGGTCGTCGGCTTCACAACAGTTGCGATTCGATGAGGAC
ACTGACCACTTCGTTGACGGTTTCCCGAAGGTCCACCAACAAGACGAACGCCCGTGTGGTAATCT
GCGAACGTCCCACCAAGGTGGTTACCGTGCCAGCCATGTCGGAGCGACGCGGAGTTTCTCTAAAG
GGGAACACGGACATCCTGTGA
SEQ ID NO:14
CG6111 Celera fly genome project protein sequence
MKCDHTLFFALFQTEQFAVLWILFTVIVLGNSAVLFVMFINKNRKSRMNYFIKQLALADLCVGLL
NVLTDIIWRITISWRAGNLACKAIRFSQVCVTYSSTYVLVAMSIDRYDAITHPMNFSKSWKRARH
LVAGAWLISALFSLPILVLYEEKLIQGHPQCWIELGSPIAWQVYMSLVSATLFAIPALIISACYA
IIVKTIWAKGSIFVPTERAGFGAAPARRASSRGIIPRAKVKTVKMTLTIVFVFIICWSPYIIFDL
LQVFGQIPHSQTNIAIATFIQSLAPLNSAANPLIYCLFSSQVFRTLSRFPPFKWFTCCCKSYRNN
SQQNRCHTVGRRLHNSCDSMRTLTTSLTVSRRSTNKTNARVVICERPTKVVTVPAMSERRGVSLK
GNTDIL
SEQ ID NO:15
Mouse TGR346a nucleic acid sequence
ATGCAGGCGCTCAACATCACCGCGGAGCAGTTTTCCCGGCTGCTGAGCGCGCACAACCTGACTCG
GGAACAGTTCATTCATCGCTATGGGCTGCGACCGCTGGTCTACACTCCGGAGCTGCCCGCGCGCG
CTAAACTGGCCTTTGCGCTGGCTGGAGCACTCATTTTTGCCCTGGCGCTCTTTGGCAACTCTCTG
GTCATCTATGTGGTGACCCGCAGCAAGGCCATGCGCACCGTCACCAACATCTTCATCTGCTCTCT
GGCACTCAGTGATCTGCTCATTGCCTTCTTCTGCATCCCCGTCACGATGCTCCAGAACATCTCCG
ACAAGTGGCTGGGTGGTGCCTTCATCTGCAAGATGGTGCCCTTCGTCCAGTCCACTGCTGTTGTG
ACGGAAATCCTCACCATGACTTGCATCGCTGTTGAGAGGCACCAAGGACTCATCCATCCTTTTAA
AATGAAGTGGCAGTACACTACCCGAAGGGCTTTCACAATCTTGGGTGTGGTCTGGTTGGCAGCCA
TCATCGTAGGATCACCCATGTGGCACGTACAACGCCTCGAGATTAAGTATGACTTCCTCTATGAG
AAAGAACATGTCTGCTGTTTGGAAGAGTGGGCCAGCCCCATGCACCAGAGAATCTACACCACCTT
CATCCTCGTCATCCTCTTCCTCCTGCCGCTTGTGGTGATGCTTGTCCTCTACAGCAAGATTGGCT
ATGAACTGTGGATCAAGAAGAGAGTTGGAGACAGTTCAGCACTTCAGACTATCCACGGGAAAGAA
ATGTCCAAAATAGCCAGGAAGAAGAAGCGGGCTGTCGTTATGATGGTGACAGTGGTGGCTCTCTT
CGCTGCGTGCTGGGCACCTTTCCATGTTGTTCACATGATGGTTGAGTACAGTAACTTTGAAAAAG
AGTATGATGATGTCACAATCAAGATGGTTTTTGCTGTTGCACAAACAATTGGCTTTTTCAACTCC
ATCTGTAATCCCTTTGTGTATGCATTTATGAATGAAAACTTCAAAAAGAATTTTTTGTCTGCGGT
TTGTTATTGCATAGTAAGAGAAACCTTCTCCCCAGGACAGAAGCCTGGAAATTCTGGGATTTCAA
TGATGCAAAAGAGAGCAAAGTTATCACGATCACAGCGTCCAGTGGCGGAAGCCAAAGGAGACTTA
TTCAGCGATGCCAACGTTGATGTCAAATTGTGTGAGCAGCCAGGGGAGAAAAGGCAACTCAAGCG
ACAGCTTGCCTTCTTTAGTTCTGAACTTTCTGAAAACTCTACTTTCGGCAGTGGACATGAACTGT
AATGATATCCTCATAGCTAATATCATTTGTATGGAAAGTTATTTTAAGCAAAGGTCAGGACTATT
TTTTTTAAATGACAAGAAGAGAAACAAGACATGTTTTCCATTTAAATGAACATAATACATAACAC
TGTAACTTTGAAAAATTATTATAACAGCTTTGTAGATGATAAAAGTAGATTTTTGAAAGTCTTCG
TACATAATAAAGCAGTGGTTTTGGCAGCAGTTTTATCCATGTAGTCAATGTAATGTGACTTTTAT
GTATTGCTACACTGGATGAAAATTATTAAAATTGTGTCATCATCCTTGAATATTAAACATCTGAA
CATCATAATGTAGTTTGTAGTGTGCTGTAAACGTTTGTAAAATCAGCCTTTGGAACTGACATCTG
TGCCATAATTAAAAAATCAAGGAGGATGAAGAATCAGGCAAGTGACA
SEQ ID NO:16
Mouse TGR346a protein sequence
MQALNITAEQFSRLLSAHNLTREQFIHRYGLRPLVYTPELPARAKLAFALAGALIFALALFGNSL
VIYVVTRSKAMRTVTNIFICSLALSDLLIAFFCIPVTMLQNISDKWLGGAFICKMVPFVQSTAVV
TEILTMTCIAVERHQGLIHPFKMKWQYTTRRAFTILGVVWLAAIIVGSPMWHVQRLEIKYDFLYE
KEHVCCLEEWASPMHQRIYTTFILVILFLLPLVVMLVLYSKIGYELWIKKRVGDSSALQTIHGKE
MSKIARKKKRAVVMMVTVVALFAACWAPFHVVHMMVEYSNFEKEYDDVTIKMVFAVAQTIGFFNS
ICNPFVYAFMNENFKKNFLSAVCYCIVRETFSPGQKPGNSGISMMQKRAKLSRSQRPVAEAKGDL
FSDANVDVKLCEQPGEKRQLKRQLAFFSSELSENSTFGSGHEL
SEQ ID NO:17
Mouse TGR346b nucleic acid sequence
ATGTCGTGGAACTTGACCGCGGAGCAGCTCTCGGCGCTGCTTCGGCTGCACAACCTGACGCGCGC
TCAGTTCATCGCGCACTATGGGCTGCGGCCACTGGTGCTCACCCCGCAGCTTCCCGCGCGCGCCA
GGCTGGCCCTCCTGCTGGTCGGCATGCTCATCTTTGCCCTGGCGCTCTTCGGCAACGCCCTGGTA
GTCTATGTGGTGACCCGCAGCAAGGCCATGCGCACCGTCACCAACATCTTCATCTGCTCCCTGGC
ACTCAGCGACCTGCTCATCGTCTTCTTCTGCATCCCGGTCACCATGCTCCAGAACGTCTCGGACA
CCTGGCTGGGGGGTGCCTTCATTTGCAAAATGGTCCCATTTGTCCAGTGCACTGCCATTGTGACA
GAAATCCTTACTATGACCTGCATTGCTGTGGAAAGGCACCAGGGACTTGTCCATCCTTTTAAAAT
GAAGCGGCAGTACACCAATCAAAGAGCTTTCACAATGCTAGGTGTGGTGTGGCTGGTGGCCATCA
TCATAGGATCACCCATGTGGCATGTGCAGCGACTTGAGATTAAGTATGACTTCCTATATGAAAAA
GAACACATCTGCTGCCTGGAAGAGTGGAGCAGCCCCGTGCACCAGAAGATCTACACCACCTTCAT
CCTTGTCACCCTCTTCCTGCTACCACTGTTGCTGCTCTCTGTCCTCTACGGGAAAATCGGTTATG
AGCTTTGGATCAAGAAAAGAATCGGGGATGGCTCAGTGCTCCGAACTATTCATGGAAAAGAAATG
TTCAAAATAGCCAGAAAGAAGAAGCGAGCTGTGATCATGATGGTGACAGTCGTGGTTCTCTTTGC
TGTGTGCTGGGCACCTTTCCACATCGTTCACATGATGATTGAATACAGTAATTTTGAAAAGGAAT
ATGATGAAGTCACAATCAAGATGATTTTTGCTATAGTGCAAATAATTGGATTTTTCAACTCCATC
TGTAATCCCATTATTTATGCACTTATGAATGAAAACTTCAAAAAAAACTTTGTGTCTGCCGTTTG
CTATTGCATTGTGAAGGAAACACCTTCTTCAGCACGGAAGCATGGAAGTTCAGGAGCTATGGTGA
TGCACAGGAGGGCAAAGTTAGCTGCAAGAGAGAATCCTGTAGAGATCAAAGGAGAAGCATTTGGG
GGCAGCAACATCGATATCAAGTGGTGTGAACAGCCAGAAAAGAAGAAGAGGAGATCAAAAGTGGC
ATCTTGTCCTCTTTAGTTCCGAATTTCTGAGAGCTCTGCTGTAGACGTGAACACTGTACCAATGT
CTTCAGAATGAGTATCTGTCATACTGTAATCGAAAGAAAATGATTTTGAGAAAAAGCCAGAGAGC
TTTCATATTAAAAATGTTGACAAACACTCAGAAGGCAGGGACAGGGGATTCAAGAGTTTAAAGTC
ATCCTTAGCTGCACGATAAGTTTGAGGATAACCTGGGCTACAAGAGACCCTGTCTCAAGAAGCCA
TAATAATTAAAACAACCATCCTTAACTAATGATAATGACAAAGTATTTTTCCATTGAAAATACAT
GTAAGCTGCAATTTTGAAAAATTATTGAACCACCCTTGTGATTAATAGATGAAGTTTAAAAAAAT
TTAAATGTGTTTTTATTGTATGTATATGGTTGGTTTACCTGTGTATATGTCTTTCAGTAACTTGT
ATAAAACTCAATGATCTCAGCTAGTAACTTTCTTCTGTGTGGTCAATGTGATATGATTTCCTATA
TATTGCTAAATTGAATG
SEQ ID NO:18
Mouse TGR346b protein sequence
MSWNLTAEQLSALLRLHNLTRAQFIAHYGLRPLVLTPQLPARARLALLLVGMLIFALALFGNALV
VYVVTRSKAMRTVTNIFICSLALSDLLIVFFCIPVTMLQNVSDTWLGGAFICKMVPFVQCTAIVT
EILTMTCIAVERHQGLVHPFKMKRQYTNQRAFTMLGVVWLVAIIIGSPMWHVQRLEIKYDFLYEK
EHICCLEEWSSPVHQKIYTTFILVTLFLLPLLLLSVLYGKIGYELWIKKRIGDGSVLRTIHGKEM
FKIARKKKRAVIMMVTVVVLFAVCWAPFHIVHMMIEYSNFEKEYDEVTIKMIFAIVQIIGFFNSI
CNPIIYALMNENFKKNFVSAVCYCIVKETPSSARKHGSSGAMVMHRRAKLAARENPVEIKGEAFG
GSNIDIKWCEQPEKKKRRSKVASCPL

1. An isolated nucleic acid encoding a G-protein coupled receptor polypeptide, the nucleic acid encoding a polypeptide comprising greater than 70% amino acid identity to an amino acid sequence of SEQ ID NO:8 or SEQ ID NO:10, or SEQ ID NO:12.

2. The isolated nucleic acid of claim 1, wherein the nucleic acid encodes a polypeptide having at least 50 contiguous amino acids of an amino acid sequence of SEQ ID NO:8, SEQ ID NO:10, or SEQ ID NO:12.

3. The isolated nucleic acid of claim 1, wherein the nucleic acid encodes a polypeptide that specifically:binds to polyclonal antibodies generated against an amino acid sequence of SEQ ID NO:8, SEQ ID NO:10, or SEQ ID NO:12.

4. The isolated nucleic acid of claim 1, wherein the nucleic acid encodes a polypeptide that has G-protein coupled receptor activity.

5. The isolated nucleic acid of claim 1, wherein the nucleic acid encodes a polypeptide comprising an amino acid sequence of SEQ ID NO:8, SEQ ID NO:10, or SEQ ID NO:12.

6. The isolated nucleic acid of claim 1,wherein the nucleic acid comprises a nucleotide sequence of SEQ ID NO:7, SEQ ID NO:9, or SEQ ID NO:11.

7. The isolated nucleic acid of claim 1, wherein the nucleic acid is amplified by primers that specifically hybridize under stringent hybridization conditions to a nucleic acid having a nucleotide sequence of SEQ ID NO:7, SEQ ID NO:9, or SEQ ID NO:11.

8. An isolated nucleic acid-encoding a G-protein coupled receptor polypeptide, wherein the nucleic acid specifically hybridizes under stringent hybridization conditions to a nucleic acid having a nucleotide sequence of SEQ ID NO:7, SEQ ID NO:9, or SEQ ID NO:11.

9. An isolated nucleic acid encoding a G-protein coupled receptor polypeptide, the polypeptide encoded by the nucleic acid comprising greater than about 70% amino acid identity to a polypeptide having an amino acid sequence of SEQ ID NO:8, SEQ ID NO:10, or SEQ ID NO:12, wherein the nucleic acid selectively hybridizes under moderately stringent hybridization conditions to a nucleotide sequence of SEQ ID NO:7, SEQ ID NO:9, or SEQ ID NO:11.

10. An isolated G-protein coupled receptor polypeptide, the polypeptide comprising greater than about 70% amino acid sequence identity to an amino acid sequence of SEQ ID NO:8, SEQ ID NO:10, or SEQ ID NO:12.

11. The isolated polypeptide of claim 10, wherein the polypeptide specifically binds to polyclonal antibodies generated against SEQ ID NO:8, SEQ ID NO:10, or SEQ ID NO:12.

12. The isolated polypeptide of claim 10, wherein the polypeptide has G-protein coupled receptor activity.

13. The isolated polypeptide of claim 10, wherein the polypeptide has an amino acid sequence of SEQ ID NO:8, SEQ ID NO:10, or SEQ ID NO:12.

14. An antibody that selectively binds to the polypeptide of claim 10.

15. An expression vector comprising the nucleic acid of claim 1.

16. A host cell transfected with the vector of claim 15.

17. An isolated nucleic acid encoding a G-protein coupled receptor polypeptide, the nucleic acid encoding a polypeptide comprising greater than 85% amino acid identity to an amino acid sequence of SEQ ID NO:16 or SEQ ID NO:18.

18. The isolated nucleic acid of claim 17, wherein the nucleic acid encodes a polypeptide having at least 50 contiguous amino acids of an amino acid sequence of SEQ ID NO:16 or SEQ ID NO:18.

19. The isolated nucleic acid of claim 17, wherein the nucleic acid encodes a polypeptide that specifically binds to polyclonal antibodies generated against an amino acid sequence of SEQ ID NO:16 or SEQ ID NO:18.

20. The isolated nucleic acid of claim 17, wherein the nucleic acid encodes a polypeptide that has G-protein coupled receptor activity.

21. The isolated nucleic acid of claim 17, wherein the nucleic acid encodes a polypeptide comprising an amino acid sequence of SEQ ID NO:16 or SEQ ID NO:18.

22. The isolated nucleic acid of claim 17, wherein the nucleic acid comprises a nucleotide sequence of SEQ ID NO:15 or SEQ ID NO:17.

23. The isolated nucleic acid of claim 17, wherein the nucleic acid is amplified by primers that specifically hybridize under stringent hybridization conditions to a nucleic acid having a nucleotide sequence of SEQ ID NO:15 or SEQ ID NO:17.

24. An isolated nucleic acid encoding a G-protein coupled receptor polypeptide, wherein the nucleic acid specifically hybridizes under stringent hybridization conditions to a nucleic acid having a nucleotide sequence of SEQ ID NO:15 or SEQ ID NO:17.

25. An isolated nucleic acid encoding a G-protein coupled receptor polypeptide, the polypeptide encoded by the nucleic acid comprising greater than about 85% amino acid identity to a polypeptide having an amino acid sequence of SEQ ID NO:16 or SEQ ID NO:18, wherein the nucleic acid selectively hybridizes under moderately stringent hybridization conditions to a nucleotide sequence of SEQ ID NO:15 or SEQ ID NO:17.

26. An isolated G-protein coupled receptor polypeptide, the polypeptide comprising greater than about 85% amino acid sequence identity to an amino acid sequence of SEQ ID NO:16 or SEQ ID NO:18.

27. The isolated polypeptide of claim 26, wherein the polypeptide specifically binds to polyclonal antibodies generated against SEQ ID NO:16 or SEQ ID NO:18.

28. The isolated polypeptide of claim 26, wherein the polypeptide has G-protein coupled receptor activity.

29. The isolated polypeptide of claim 26, wherein the polypeptide has an amino acid sequence of SEQ ID NO:16 or SEQ ID NO:18.

30. An antibody that selectively binds to the polypeptide of claim 26.

31. An expression vector comprising the nucleic acid of claim 17.

32. A host cell transfected with the vector of claim 31.

33. A method for identifying a compound that modulates signal transduction, the method comprising the steps of:
(i) contacting the compound with-a polypeptide comprising greater than 70% amino acid sequence identity to the amino acid sequence of SEQ ID NO:2, SEQ ID NO:4, SEQ ID NO:6, SEQ ID NO:8, SEQ ID NO:10, SEQ ID NO:16 and SEQ ID NO:18; and
(ii) determining the functional effect of the compound upon the polypeptide.

34. The method of claim 33, wherein the polypeptide has G-protein coupled receptor activity.

35. The method of claim 33, wherein the polypeptide comprises greater than 70% amino acid sequence identity to the amino acid sequence of SEQ ID NO:8 or SEQ ID NO:10 or greater than 85% amino acid sequence identity to the amino acid sequence of SEQ ID NO:16 and SEQ ID NO:18.

36. The method of claim 33, wherein the polypeptide is linked to a solid phase.

37. The method of claim 33, wherein the functional effect is determined by measuring changes in intracellular cAMP, IP3, or Ca²⁺.

38. The method of claim 33, wherein the functional effect is determined by measuring binding of the compound to the polypeptide.

39. The method of claim 33, wherein the polypeptide comprises an amino acid sequence of SEQ ID NO:8, SEQ ID NO:10, SEQ ID NO:16 and SEQ ID NO:18.

40. The method of claim 33, wherein the polypeptide is expressed in a cell or cell membrane.

41. The method of claim 40, wherein the cell is selected from the group consisting of an adipocyte cell, a spleen cell, a colon cell, a kidney cell, a neuron, a skeletal muscle cell, an ocular cell, a retina cell, a hypothalamus cell, and a tongue cell.

42. A method of identifying a mammal having a TGR-associated disorder, comprising detecting-a TGR nucleic acid molecule in a sample from the mammal, wherein said TGR nucleic acid molecule is a nucleic acid comprising greater than 70% nucleic acid sequence identity to the nucleic acid sequence of SEQ ID NO:1, SEQ ID NO:3, SEQ ID NO:5, SEQ ID NO:7, SEQ ID NO:9, SEQ ID NO:15 and SEQ ID NO:17, and wherein abnormal expression of the TGR nucleic acid molecule in the sample indicates that the mammal has a TGR-associated disorder.

43. The method of claim 42, wherein the TGR nucleic acid molecule comprises greater than 70% nucleic acid sequence identity to the nucleic acid sequence of SEQ ID NO:7, SEQ ID NO:9, SEQ ID NO:15 and SEQ ID NO:17.

44. A method of identifying a mammal having a TGR-associated disorder, comprising detecting a TGR polypeptide in a sample from the mammal, wherein the TGR polypeptide comprises greater than 70% amino acid sequence identity to the amino acid sequence of SEQ ID NO:2, SEQ ID NO:4, SEQ ID NO:6, SEQ ID NO:8, SEQ ID NO:10, SEQ ID NO:16 and SEQ ID NO:18, and wherein abnormal expression of the TGR polypeptide in the sample indicates that the mammal has a TGR-associated disorder.

45. The method of claim 44, wherein the TGR polypeptide comprises greater than 70% amino acid sequence identity to the amino acid sequence of SEQ ID NO:8, SEQ ID NO:10, SEQ ID NO:16 and SEQ ID NO:18.

46. A method of treating or preventing a TGR-associated disorder, comprising administering a therapeutically effective amount of a modulator identified using the method of claim 33 to a mammal in need thereof.

47. A method of treating retinitis pigmentosa, the method comprising the step of administering to a patient a compound that modulates the activity of TGR60.

48. A method of regulating circadian rhythms, the method comprising the step of administering to a patient a compound that modulates the activity of TGR60.
